(19) United States
(12) Patent Application Publication
(10) Pub. No.: US 2012/0078017 A1
(43) Pub. Date: Mar. 29, 2012

(54) METHODS FOR PREVENTING AND TREATING RADIATION-INDUCED EPITHELIAL DISORDERS

(76) Inventors: John C. Alverdy, Glenview, IL (US); Olga Zaborina, Brookfield, IL (US)

(21) Appl. No.: 13/259,313

(22) PCT Filed: Mar. 23, 2010
(86) PCT No.: PCT/US2010/028349

§ 371 Date: Nov. 14, 2011

Related U.S. Application Data
(60) Provisional application No. 61/162,544, filed on Mar. 23, 2009.

(51) Int. Cl. C07C 43/23; C07C 43/11

(52) U.S. Cl. 568/609; 568/623

(57) ABSTRACT

The present invention provides methods of protecting irradiated eukaryotic cells such as irradiated mammalian epithelial cells, from the deleterious effect of microbial pathogens such as Pseudomonas aeruginosa. The invention also provides methods of protecting irradiated organisms from such deleterious effects, resulting in reduced mortality and morbidity. Further provided are kits containing relatively high molecular weight biocompatible polymers such as polyethylene glycol, optionally supplemented with a protective polymer such as dextran and/or essential pathogen nutrients such as L-glutamine, along with instructions for administration to organisms to be exposed to radiation.

This invention was made with government support under R01-GM62344 awarded by the National Institutes of Health. The government has certain rights in the invention.

### FIELD OF INVENTION

The present invention relates to materials and methods for preventing or treating microbe-mediated epithelial disorders, such as radiation-induced epithelial cell disorders.

### BACKGROUND

Microbe-mediated epithelial disorders, or abnormal conditions, present a significant threat to the health of man and animals, imposing a burden on healthcare systems worldwide. One example of such disorders, gut-derived sepsis, is a major cause of mortality among organisms, such as human patients, that suffer from any of a variety of diseases, disorders or afflictions, such as burn injuries, neonatal enterocolitis, severe neutropenia, inflammatory bowel disease, and organ rejection following transplantation. Additionally, gastrointestinal toxicity leading to sepsis following abdominal radiation continues to pose a significant health risk both from abdominal radiotherapy to treat cancer and accidental exposure. The intestinal tract reservoir has long been recognized to be a potentially lethal focus of bacterial-mediated sepsis in, e.g., critically ill, hospitalized patients. The ability of microbial pathogens such as the Pseudomonads (e.g., Pseudomonas aeruginosa) to perturb the regulatory function of the intestinal epithelial barrier may be a defining characteristic among opportunistic organisms capable of causing gut-derived sepsis. In many of these infections, Pseudomonas aeruginosa has been identified as the causative pathogen. Significantly, the intestinal tract has been shown to be the primary site of colonization of opportunistic pathogens such as P. aeruginosa.

Conventional therapeutic approaches to the prevention or treatment of microbe-mediated epithelial disorders such as gut-derived sepsis have met with incomplete success. Antibiotic-based approaches are compromised by the difficulty in tailoring antibiotics to the intestinal pathogen in a manner that does not impact the remaining intestinal flora. In addition, many of the intestinal pathogens, as typified by P. aeruginosa, often become resistant to antibiotic challenges, resulting in a costly, ongoing and incompletely successful approach to prevention or treatment. Problems also plague immunotherapeutic approaches. Particularly, many intestinal pathogens such as P. aeruginosa, are immunoevasive, rendering such approaches minimally effective.

Another approach to the prevention or treatment of disorders such as gut-derived sepsis is intestinal lavage. In the past several years, intestinal lavage using polyethylene glycol (PEG) solutions has been attempted, with some anecdotal reports suggesting that PEG may show some promise in treating gut-derived sepsis across a variety of clinical and experimental circumstances. The PEG in these solutions has an average molecular weight of 3,500 daltons and the solutions are commercially available (e.g., Golytely). The mechanisms by which these relatively low molecular weight (LMW) solutions of PEG provide a therapeutic benefit in treating or preventing gut-derived sepsis is unknown. Typically, these solutions are used to wash or flush the intestinal tract of organisms at risk of developing, or suffering from, gut-derived sepsis. As a result of administering these LMW PEG solutions to the intestinal tract, there is a variable change in the floral composition of the treated intestine depending on the method of administration, concentration, and the molecular weight of the compounds used. For example, solutions having concentrations of PEG higher than about 20% can result in a microbiocidal action resulting in the elimination of potentially protective microorganisms in the intestinal tract of a stressed host. Also, solutions of low molecular weight PEG can lose their efficacy in attenuating the virulence capacity of certain pathogenic organisms, despite preserving them. Therefore, a need exists in the art for a solution that inhibits microbial virulence expression (the harmful properties of a microbe) while not killing the microbe or neighboring microbes, thereby providing the benefit of preserving the natural ecosystem of the intestinal microflora. For example, preservation of the native floral composition would provide competition for opportunistic pathogens that might otherwise colonize the intestine.

Concomitant with a change in floral composition is a change in the physiology of the organism. These physiological changes may be monitored by assaying any number of characteristic enzymatic activities, such as lactate dehydrogenase levels. Consequently, LMW PEG treatments of the intestine produce significant changes in the physiology of the treated organisms, with unpredictable, and thus potentially deleterious, longer-term consequences for the health and well-being of the treated organism. Moreover, such treatments provoke physically demanding reactions in the form of massive intestinal voiding in critically ill organisms such as hospitalized human patients.

Another type of epithelial cell disorder, mediated in part by microbes, is radiation-induced damage to epithelial cells exposed to microbes, leading to inflammation, loss of epithelial cell (e.g., intestinal) barrier function and bacterial dissemination, in turn leading to severe and even lethal sepsis. The mechanism by which radiation, e.g., abdominal radiation, causes such effects on cells (e.g., epithelial cells) is complex. Radiation has been shown to affect various components of the local intestinal response to injury and microbial invasion that includes rapid depletion of mucus, immune impairment, oxidant-mediated epithelial cell injury, and radiation-induced epithelial cell apoptosis. In addition, radiation exposure itself causes a major shift in the intestinal microflora whereby radiation effects directly destroy large densities of probiotic bacteria that play a key role in maintaining normal function of the intestinal immune system and colonization resistance to transient microbes. The results of the effects of radiation on the intestinal epithelium, its overlying mucus layer, its underlying immune repertoire, and its response in the context of a shifting microbial flora, illustrate a need for a multi-pronged approach to treat and/or prevent radiation-induced loss of epithelial cell barrier function and bacterial dissemination. Although recent reports have provided striking evidence that radiation-induced intestinal injury and subsequent mortality can be prevented by epithelial toll receptor activation, these promising results in animals do not take into account the common observation that in patients, fatal bacteremias are often caused by virulent and resistant nosocomial pathogens such as Pseudomonas aeruginosa. Pseudomonas aeruginosa is the most common pathogen to cause fatal gut-derived sepsis after bone marrow transplantation, radiation induced enteritis, and chemotherapy. The antibiotic resistance profile of this immunoelusive pathogen makes it particularly problematic in these clinical circumstances. Patients, in contrast to laboratory animals, become rapidly colonized with P. aeruginosa due to antibiotic selection and cross contamination. In hospitalized patients, the intestinal tract is the primary site of colonization of P. aeruginosa where as many as 50% of patients' feces are culture-positive within five days of hospitalization and antibiotic exposure. As a consequence, strategies to limit the intestinal damage from radiation in terms of loss of epithelial barrier function and fatal gut-derived sepsis need to consider that the shift in the human microflora in these clinical situations differs substantially from that of laboratory animals.

Thus, there remains a need in the art to provide a composition effective in preventing, or treating, a microbe-mediated epithelial disorder (e.g., gut-derived sepsis) and/or a symptom associated with such a disorder, along with methods for achieving such benefits, without creating the potential for further complications through significant alteration of the physiology of the treated organism.

### SUMMARY OF THE INVENTION

The present invention satisfies at least one of the aforementioned needs in the art by providing a high molecular weight (HMW) polyethylene glycol composition that provides effective protection to irradiated cells, tissues and organisms against the deleterious effect of a microbial pathogen. Also provided are uses of the HMW polyethylene glycol (HMW PEG) in the preparation of medicaments for the methods disclosed herein. Exemplary microbial pathogens are bacterial pathogens such as Pseudomonas aeruginosa. The HMW PEG inhibits or prevents contact of such pathogens as P. aeruginosa with an epithelial surface, such as an intestinal epithelial surface. In addition, high molecular weight PEG suppresses virulence expression in these pathogens (e.g., P. aeruginosa) responsive to a variety of signals that involve quorum sensing signaling networks. Further, HMW PEGs interact with lipid rafts in the membranes of epithelial cells and alter apoptotic signaling pathways within the cells in a protective manner. The ability of HMW PEGs to interdict at the infectious interface between the microbial pathogen and the host epithelium provides an approach to ameliorating or eliminating untoward consequences of radiotherapy. Importantly, treatments with HMW PEGs would be cost effective and relatively simple to perform on human patients as well as a variety of other organisms such as agriculturally significant livestock (e.g., cattle, pigs, sheep, goats, horses, chickens, turkeys, ducks, geese, and the like), pets, and zoo animals.

One aspect of the invention provides a method for treating a mammalian epithelial cell exposed to radiation comprising administering a therapeutically effective amount of a high molecular weight polyethylene glycol (HMW PEG) compound to the mammalian epithelial cell. In some embodiments of the method, the mammalian epithelial cell is further exposed to an intestinal pathogen, such as a Pseudomonad (e.g., Pseudomonas aeruginosa). In the method, the HMW PEG compound may have an average molecular weight selected from the group consisting of at least 1,000 daltons, at least 5,000 daltons, at least 8,000 daltons, at least 12,000 daltons and at least 15,000 daltons. A variety of structures of HMW PEG meeting the minimum average molecular weight criterion set forth above are contemplated, including a HMW PEG compound that comprises at least two hydrocarbon chains attached to a hydrophobic core, wherein each hydrocarbon chain has an average molecular weight of at least 40 percent of the HMW PEG compound, and wherein the hydrophobic core comprises a ring structure. The method comprehends conditions wherein the HMW PEG is administered within a relatively short time frame after irradiation, and specifically comprehends administration of HMW PEG within five minutes of exposing the cell to radiation.

Another aspect according to the disclosure is a method of treating a microbial pathogen-induced disorder of an irradiated mammalian epithelial cell comprising administering a therapeutically effective amount of a high molecular weight polyethylene glycol (HMW PEG) compound to a mammalian epithelial cell exposed to radiation. A therapeutically effective amount of HMW PEG will vary depending on known variables such as the age, weight, general health of the patient or animal subject, and a therapeutically effective amount is readily determinable using routine procedures, as would be known in the art. In some embodiments of the method, the HMW PEG is administered prior to irradiation of the mammalian epithelial cell. The method comprehends the administration of HMW PEGs having the above-defined minimal average molecular weights, and comprehends an embodiment in which the HMW PEG having at least two hydrocarbon chains and a hydrophobic core, as described above, is administered. The method specifically comprehends treatment wherein the microbial pathogen is Pseudomonas aeruginosa.

Another aspect of the invention is a method of treating a mammalian epithelial cell with radiotherapy comprising administering a therapeutically effective combination of radiation and a high molecular weight polyethylene glycol (HMW PEG) compound. As in other aspects according to the disclosure, the HMW PEG may have an average molecular weight selected from the group consisting of at least 1,000 daltons, at least 5,000 daltons, at least 8,000 daltons, at least 12,000 daltons and at least 15,000 daltons. In some embodiments, the radiation is selected from the group consisting of gamma radiation and X-ray radiation.

Yet another aspect according to the disclosure is a method of protecting a mammalian epithelial cell from radiation-induced damage, comprising administering a prophylactically effective amount of a high molecular weight polyethylene glycol (HMW PEG) compound to an epithelial cell at risk of radiation-induced damage. Again, the HMW PEG compound may have an average molecular weight selected from the group consisting of at least 1,000 daltons, at least 5,000 daltons, at least 8,000 daltons, at least 12,000 daltons and at least 15,000 daltons.

Still another aspect is a method of protecting an irradiated mammalian epithelial cell from microbial pathogen-induced damage comprising administering a therapeutically effective amount of a high molecular weight polyethylene glycol (HMW PEG) compound to an irradiated mammalian epithelial cell. The HMW PEG compound may have an average molecular weight selected from the group consisting of at least 1,000 daltons, at least 5,000 daltons, at least 8,000 daltons, at least 12,000 daltons and at least 15,000 daltons. In some embodiments, the microbial pathogen is Pseudomonas aeruginosa.

Yet another aspect is drawn to a method of preventing sepsis in a mammal having abdominal radiation comprising administering a therapeutically effective amount of a high molecular weight polyethylene glycol (HMW PEG) compound to the abdominal region of the mammal. In some embodiments, the HMW PEG is administered prior to irradiation of the abdominal region; in other embodiments, the HMW PEG is administered following irradiation. In some embodiments, the HMW PEG is administered continuously or in a plurality of batches for at least forty-eight hours.

In each of the foregoing aspects according to the disclosure, the HMW PEG may be administered by any route known in the art, such as a route selected from the group consisting of topical administration, enteric administration and parenteric administration. More particularly, any of the foregoing methods may involve HMW PEG administration epicutaneously, inhalationally, intranasally, by eye drops, by ear drops, orally, by gastric feeding tube, by duodenal feeding tube, by gastrostomy, by enema, by suppository, by gastric lavage, by pulmonary lavage, by colonic lavage, by subpalpebral lavage, intravenously, intraarterially, intramuscularly, subcutaneously, by osseous infusion, intrathecally, intradermally, transdermally, transmucosally, by insufflation, or intravitreally. Also in each of the foregoing aspects according to the disclosure, the HMW PEG may have at least two hydrocarbon chains wherein each chain has an average molecular weight of at least 40 percent of the HMW PEG compound, and a hydrophobic core, wherein the hydrophobic core may include one or more aromatic or non-aromatic rings.

Other features and advantages of the present invention will be better understood by reference to the following detailed description, including the drawing and the examples.

### BRIEF DESCRIPTION OF THE DRAWING

FIG. 1 provides mortality rates in mice at 48 hours subjected to either sham laparotomy or 30% surgical hepatectomy followed by direct injection of P. aeruginosa PA27853 into the cecum. Mice underwent a 30% bloodless left lobe hepatectomy immediately followed by direct cecal injection of 1×10⁷ cfu/ml of PA27853. Each group contained 7 mice. Control mice underwent sham laparotomy followed by injection of equal amounts of PA27853 into the cecum. For mice in the PEG groups, 1×10⁷ cfu/ml of PA27853 was suspended in either PEG 3.35 (LMW PEG 3,350) or PEG 15-20 (HMW PEG 15,000 to 20,000 daltons) prior to cecal injection. Dose response curves for PEG 15-20 are seen in panel b. a. A statistically significant protective effect of PEG 15-20 was determined by the Fisher Exact Test (P<0.001). b. The minimum protective concentration of PEG 15-20 was determined to be 5% (P<0.05). c. Quantitative bacterial cultures of cecal contents (feces), washed cecal mucosa, liver, and blood 24 hours following 30% surgical hepatectomy and direct cecal injection of 1×10⁷ cfu/ml of PA27853. One-way ANOVA demonstrated a statistically significant increase in bacterial counts in cecal contents, mucosa, liver, and blood in mice following hepatectomy (P<0.001). A significant decrease (P<0.05) in the liver and blood bacterial counts was observed for PEG 3350, while PEG 15-20 completely prevented PA27853 from disseminating to the liver and blood of mice.

FIG. 2 shows the protective effect of PEG 15-20 against PA27853-induced epithelial barrier dysfunction as assessed by transepithelial electrical resistance (TEER). a. Data represent the mean±SEM % maximal fall in TEER from baseline of triplicate cultures (n=7) observed during 8 hours of apical exposure to 1×10⁷ cfu/ml of PA27853. A statistically significant decrease in TEER was demonstrated (one-way ANOVA (P<0.001)) in Caco-2 cells exposed to PA27853. A statistically significant protective effect on the fall in TEER induced by PA27853 was demonstrated for PEG 15-20 (P<0.001). b. Image of Caco-2 cells in the presence of PEG 3.35 and apical exposure to PA27853. Images taken after 4 hours of co-culture demonstrated loss of monolayer integrity with cells floating 30-40 microns above the cell scaffolds and displaying adherence of PA27853 to cell membranes. c. Caco-2 cells apically exposed to PA27853 after 4 hours in the presence of PEG 15-20 showed no evidence of floating cells in any of the planes examined.

FIG. 3 illustrates the inhibitory effect of PEGs on PA-I expression in PA27853. a. Western blot analysis. Exposure of PA27853 to 1 mM of the quorum-sensing signaling molecule C4-HSL resulted in a statistically significant increase (P<0.001 one-way ANOVA) in PA-I protein expression that was partially inhibited in the presence of 10% PEG 3.35 and significantly more inhibited with 10% PEG 15-20. a′. The minimum inhibitory concentration of PEG 15-20 on C4-HSL induced PA-I expression was 5% (P<0.01). b. Electron microscopy of individual bacteria cells exposed to C4-HSL in the presence and absence of PEGs, demonstrated that C4-HSL caused a morphological change in the shape and pili expression of P. aeruginosa. The C4-HSL-induced morphological effect was completely eliminated in the presence of PEG 15-20, but not PEG 3.35. A halo-type effect can be seen surrounding PA27853 exposed to PEG 15-20. c. Northern hybridization. Exposure of PA27853 to 0.1 mM of C4-HSL resulted in a statistically significant increase (P<0.001 one-way ANOVA) in PA-I mRNA expression that was greatly inhibited with 10% PEG 15-20. d. The increase in PA-I mRNA levels induced by 4 hours exposure to Caco-2 cell was inhibited in the presence of PEG 15-20, but not PEG 3.35 (P<0.001 one-way ANOVA).

FIG. 4 shows the effect of PEG solutions on bacterial membrane integrity and growth patterns of PA27853. a. The effect of the two PEG solutions on bacterial membrane integrity was assessed by a staining method consisting of SYTO 9 and propidium iodide. Neither PEG solution had any effect on bacterial membrane permeability. b. PA27853 growth patterns appeared identical in the two PEG solutions relative to the PEG-free TSB medium (control).

FIG. 5 presents Atomic Force Microscopy (AFM) images of Caco-2 cells and bacterial cells exposed to PEGs. a-c. AFM images of Caco-2 cells in the presence of medium alone (a), medium with PEG 3.35 (b), and medium with PEG 15-20. PEG 3.35 was seen to form a smooth carpet over the Caco-2 cells (b), whereas PEG 15-20 formed a more topographically defined covering (c). d-f. AFM images of PA27853 in PEG 3.35 and PEG 15-20. PEG 3.35 formed a smooth envelope around individual bacterial cells (e) whereas PEG 15-20 not only tightly hugged the individual cells (f), but also increased the polymer/bacterial diameter (g,h), thereby distancing individual bacteria from one another.

FIG. 6 shows the effect of PEG solution on the dispersion/clumping pattern of PA27853. The dispersion pattern of bacterial cells in dTC3 dishes was observed directly with an Axiovert 100 TV fluorescence inverted microscope using DIC and GFP fluorescence filter, at an objective magnification of 63×. Temperature was adjusted with a Bioptechs thermostat temperature control system. Tungsten lamps (100 V) were used for both DIC and the GFP excitation. The 3D imaging software (Slidebook) from intelligent Imaging Innovations was used to image the bacterial cell dispersion pattern in the Z plane using the GFP filter. Uniformly dispersed planktonic P. aeruginosa cells in the medium without Caco-2 cells were seen on DIC image (6a₁) and Z plane reconstruction (6a₂). In the presence of Caco-2 cells, bacterial cells developed a clumped appearance (6b₁) and were seen adherent to the Caco-2 cells (6b₂). 10% PEG 3.35 decreased the motility of bacteria and induced immediate formation of mushroom-shaped bacterial microcolonies (6c₁) adhering to the bottom of the well (6c₂). In the presence of Caco-2 cells, bacterial microcolonies were on the order of 8 microns above the plane of the epithelial cells (6d_{1,2}). 10% PEG 15-20 greatly diminished the motility of P. aeruginosa cells. Nevertheless, for the first 0.5-1 hours of incubation in PEG 15-20-containing medium, bacterial cells formed spider-shaped microcolonies that were close to the bottom of the well (6e_{1,2}). Within several hours, spider leg-shaped microcolonies occupied the entire space/volume of the medium (not shown). In the presence of Caco-2 cells, P. aeruginosa cells lost the spider-like configuration and were seen elevated high above the plane of the epithelium (30-40 microns) (6f_{1,2}).

FIG. 7 illustrates that PEG 15-20 significantly decreased radiation-induced gut-derived sepsis in mice. (A) Experimental design and time course of protocol; (B) Kaplan-Mayer survival curves demonstrating the protective effect of oral PEG 15-20 (p<0.001) n=10/group.

FIG. 8 reveals the distribution and retention of PEG 15-20 in the mouse intestine. (A) Xenogen IVIS 200 images of live mice drinking (i) 5% dextrose or (ii) 5% dextrose plus 1% fluorescein-labeled PEG; Xenogen images of expelled feces mice drinking (iii) 5% dextrose versus (iv) 5% dextrose+1% fluorescein-labeled PEG. (B) fluorescent microscopy of cecum and ascending colon in mice drinking 5% dextrose (control) or 5% dextrose plus 1% fluorescein-labeled PEG (Fl-PEG) (SZX16 Olympus stereomicroscope). (C) Atomic force microscopy (AFM) images of midgut segments of intestine of mice drinking 5% dextrose (i) versus 5% dextrose+1% PEG 15-20 (ii). (D) AFM height deflection measurements between groups n=3, *p<0.05.

FIG. 9 shows that PEG 15-20 attenuates radiation-induced apoptosis in cultured intestinal epithelial cells and mouse epithelium. A. Experimental protocol for cell analysis. (B) Results of cytoplasmic histone-associated DNA fragment cell death ELISA in IEC-18 cells between groups ((n=5/group, *p<0.005). (C) Nuclear DNA fragmentation in IEC-18 cells detected by TUNEL assay: (i) control cells; (ii) cells treated with PEG 15-20 only; (iii) radiated cells, 5 Gy; (iv) radiated cells with 1 hour pre-treatment with 5% PEG. (D) TUNEL assay of intestinal epithelial cross-sections from (i) control mice, (ii) irradiated mice drinking 5% dextrose, and (iii) mice irradiated with 13 Gy and drinking 5% dextrose+1% PEG 15-20. White arrows show nuclear DNA fragmentation in crypt cells.

FIG. 10 depicts p53 expression in IEC-18 cells and in mouse intestine. (A) Immunoblotting of IEC-18 cells using anti-p53 antibodies and anti-actin antibody (values are mean±SD n=3, †p<0.005, *p<0.01). (B) Mean values of the ratio of p53-to-nuclear staining between treatment groups (mean±SD, n=10, *p<0.05). Representative Images of method of evaluation of p53 in cross-sections from (C) control, (D) radiated, and (E) radiated mice drinking PEG.

FIG. 11 shows p21 expression in IEC-18 cells and in intestinal epithelium in mice. (A) Immunoblotting of IEC-18 cells using anti-p21 antibodies and anti-actin antibody (values are mean±SD n=3, †p<0.005, *p<0.01). (B) Mean values of the ratio of p21-to-nuclear staining between treatment groups (mean±SD, n=10, *p<0.05). Representative Images of method of evaluation of p21 in cross-sections from (C) control, (D) radiated, and (E) radiated mice drinking PEG.

FIG. 12 illustrates the effect of PEG 15-20 on P. aeruginosa adherence to cultured intestinal epithelial cells and virulence as judged by expression of the epithelial barrier-disrupting protein PA-I lectin/adhesin. (A) Z-plane reconstructions of multiple stacked images of P. aeruginosa PAO1/EGFP demonstrating the spatial orientation of P. aeruginosa in relation to the IEC-18 cell surface of (i) non-irradiated cells, (ii) 5 Gy irradiated cells, and (iii) 5 Gy irradiated cells pre-treated with PEG 15-20. (B) Expression of the PA-I lectin/adhesin in P. aeruginosa PAO1/lecA::lux as measured by luminescence during direct contact of bacteria with IEC-18 cells. (C) Expression of PA-I lectin/adhesin in P. aeruginosa PAO1/lecA::lux exposed to conditioned media collected from IEC-18 cells following various treatments.

FIG. 13 illustrates the effect of PEG 15-20 on intestinal epithelial cell monolayer repair. Mechanically damaged IEC-18 monolayers (see Example 10) were treated with 5% PEG 15-20 (PEG), 50 ng/ml Epidermal Growth Factor (EGF), or a combination of both (PEG+EGF) for 24 hours or treated for 2 minutes, washed, then incubated without treatment for 24 hours (PEG 2 minutes, EGF 2 minutes and PEG+EGF 2 minutes) before monolayer repair was measured.

### DETAILED DESCRIPTION OF INVENTION

The invention provides products and methods that collectively present simple and economical approaches to the treatment and/or prevention of a variety of radiation-induced microbe-mediated epithelial disorders, i.e., abnormal conditions and diseases, that afflict many mammals, including humans. By administering high molecular weight polar polymers such as HMW polyethylene glycol to an animal in need, including those at risk, any of a number of health- or life-threatening abnormal conditions, i.e., epithelial disorders and diseases, including gut-derived sepsis, can be treated with minimal cost and minimal training of practitioners. Without wishing to be bound by theory, the benefits provided by the invention are consistent with the principle that microbe-mediated epithelial disorders can be successfully prevented, ameliorated or treated by facilitating an environment conducive to the survival of such microbes. An understanding of the following more detailed description of the invention is facilitated by initially establishing the following meanings for terms used in this disclosure.

An “abnormal condition” is broadly defined to include mammalian diseases, mammalian disorders and any abnormal state of mammalian health that is characterized by an epithelial surface at risk of developing a microbial-mediated disorder. The abnormal conditions characterized by an epithelial surface at risk of developing a microbial-mediated disorder include conditions in which the epithelial surface has developed a microbial-mediated disorder. Exemplary conditions include human diseases and human disorders requiring, or resulting from, medical intervention, such as a burn injury, neonatal enterocolitis, severe neutropenia, inflammatory bowel disease, enteropathy (e.g., of the critically ill), transplant (e.g., organ) rejection, and gastrointestinal toxicity leading to sepsis following abdominal radiation.

“Burn injury” means damage to mammalian tissue resulting from exposure of the tissue to heat, for example in the form of an open flame, steam, hot fluid, and a hot surface.

“Severe neutropenia” is given its ordinary and accustomed meaning of a marked decrease in the number of circulating neutrophils.

“Transplant rejection” refers to any development of transplanted material (e.g., an organ) recognized as being associated with ultimate rejection of that material by the host organism.

“Administering” is given its ordinary and accustomed meaning of delivery by any suitable means recognized in the art. Exemplary forms of administering include oral delivery, anal delivery, direct puncture or injection, topical application, and spray (e.g., nebulizing spray), gel or fluid application to an eye, ear, nose, mouth, anus or urethral opening.

An “effective dose” is that amount of a substance that provides a beneficial effect on the organism receiving the dose and may vary depending upon the purpose of administering the dose, the size and condition of the organism receiving the dose, and other variables recognized in the art as relevant to a determination of an effective dose. The process of determining an effective dose involves routine optimization procedures that are within the skill in the art.

An “animal” is given its conventional meaning of a non-plant, non-protist living being. A preferred animal is a mammal, such as a human.

In the context of the present disclosure, a “need” is an organismal, organ, tissue, or cellular state that could benefit from administration of an effective dose to an organism characterized by that state. For example, a human at risk of developing gut-derived sepsis, or presenting a symptom thereof, is an organism in need of an effective dose of a product, such as a pharmaceutical composition, according to the present invention.

“Average molecular weight” is given its ordinary and accustomed meaning of the arithmetic mean of the molecular weights of the components (e.g., molecules) of a composition, regardless of the accuracy of the determination of that mean. For example, polyethylene glycol, or PEG, having an average molecular weight of 3.5 kilodaltons may contain PEG molecules of varying molecular weight, provided that the arithmetic mean of those molecular weights is determined to be 3.5 kilodaltons at some level of accuracy, which may reflect an estimate of the arithmetic mean, as would be understood in the art. Analogously, PEG 15-20 means PEG whose molecular weights yield an arithmetic mean between 15 and 20 kilodaltons, with that arithmetic mean subject to the caveats noted above. These PEG molecules include, but are not limited to, simple PEG polymers. For example, a plurality of relatively smaller PEG molecules (e.g., 7,000 to 10,000 daltons) may be joined, optionally with a linker molecule such as a phenol, into a single molecule having a higher average molecular weight (e.g., 15,000 to 20,000 daltons).

“Cell membrane integrity” means the relative absence of functionally significant modifications of a cell membrane as a functional component of a living cell, as would be understood in the art.

“Detectably altered” is given its ordinary and accustomed meaning of a change that is perceivable using detection means suitable under the circumstances, as would be understood in the art.

“Growth pattern” refers collectively to the values of those properties of a cell, or group of cells (e.g., a population of cells), that are recognized in the art as characterizing cell growth, such as the generation or doubling time of the cell, the appearance of topography of a nascent group of cells, and other variables recognized in the art as contributing to an understanding of the growth pattern of a cell or group of cells.

“Inhibiting” is given its ordinary and accustomed meaning of inhibiting, reducing or preventing. For example, inhibiting morphological change means that morphological change is made more difficult or prevented entirely.

“PA-I, or PA-I lectin/adhesin, expression” means the production or generation of an activity characteristic of PA-I lectin/adhesin. Typically, PA-I lectin/adhesin expression involves translation of a PA-I lectin/adhesin-encoding mRNA to yield a PA-I lectin/adhesin polypeptide having at least one activity characteristic of PA-I lectin/adhesin. Optionally, PA-I lectin/adhesin expression further includes transcription of a PA-I lectin/adhesin-encoding DNA to yield the aforementioned mRNA.

“Epithelium-induced activation” refers to an increase in the activity of a given target (e.g., PA-I lectin/adhesin) through direct or indirect influence of an epithelial cell. In the context of the present invention, for example, epithelium-induced activation of PA-I lectin/adhesin refers to an increase in that polypeptide's activity or expression attributable to the indirect influence of an epithelium manifested through the direct contact of an epithelial cell or cells with an intestinal pathogen.

“Morphological change” is given its ordinary and accustomed meaning of an alteration in form.

“Intestinal pathogen” means a pathogenic microbe capable of causing, in whole or part, gut-derived sepsis in an animal such as a human. Intestinal pathogens known in the art are embraced by this definition, including gram negative bacilli such as the Pseudomonads (e.g., Pseudomonas aeruginosa).

“Ameliorating” means reducing the degree or severity of, consistent with its ordinary and accustomed meaning.

“Pathogenic quorum” means aggregation or association of a sufficient number of pathogenic organisms (e.g., P. aeruginosa) to initiate or maintain a quorum sensing signal, as would be known in the art.

“Interaction” is given its ordinary and accustomed meaning of interplay, as in the interplay between or among two or more biological products, such as molecules, cells, and the like.

“Transepithelial Electrical Resistance,” or TEER, is given the meaning this phrase has acquired in the art, which refers to a measurement of electrical resistance across epithelial tissue, which is non-exclusively useful in assessing the status of tight junctions between epithelial cells in an epithelial tissue.

“Adherence” is given its ordinary and accustomed meaning of physically associating for longer than a transient period of time.

“Topographically asymmetrical” refers to an image, map or other representation of the surface of a three-dimensional object (e.g., a cell) that is not symmetrical.

“Atomic force microscopy,” also known as scanning force microscopy, is a technique for acquiring a high-resolution topographical image of a substance by having a cantilevered probe traverse the surface of a sample in a raster scan and using highly sensitive means for detecting probe deflections, as would be understood in the art.

“Pharmaceutical composition” means a formulation of compounds suitable for therapeutic administration, to a living animal, such as a human patient. Preferred pharmaceutical compositions according to the invention comprise a solution balanced in viscosity, electrolyte profile and osmolality, comprising an electrolyte, dextran-coated L-glutamine, dextran-coated inulin, lactulase, D-galactose, N-acetyl D-galactosamine and 5-20% PEG (15,000-20,000).

“Adjuvants,” “carriers,” or “diluents” are each given the meanings those terms have acquired in the art. An adjuvant is one or more substances that serve to prolong or enhance the immunogenicity of a co-administered immunogen. A carrier is one or more substances that facilitate the manipulation, such as by translocation of a substance being carried. A diluent is one or more substances that reduce the concentration of, or dilute, a given substance exposed to the diluent.

“HMW PEG” refers to relatively high molecular weight PEG defined as having an average molecular weight greater than 3.5 kilodaltons. Preferably, HMW PEG has an average molecular weight greater than 1 kilodalton and, in particular embodiments, HMW PEG has an average molecular weight that is at least 5 kilodaltons, at least 8 kilodaltons, at least 12 kilodaltons, at least 15 kilodaltons, and between 15 and 20 kilodaltons. In one embodiment, the HMW PEG has at least two hydrocarbon chains, with each chain having an average molecular weight of at least 40 percent of the HMW PEG and a hydrophobic core with that core having a ring structure, such as 1-4 rings with each ring having 5 or 6 ring carbons and including, but not being limited to, aromatic rings.

Additionally, “HMW PEG” compounds include HMW PEG derivatives wherein each such derivative compound contains an HMW PEG compound as a moiety to which is attached at least one additional functional group. Thus, “HMW PEG” compounds include underivatized HMW PEG compounds and HMW PEG derivative compounds. Preferred HMW PEG derivatives are cationic polymers. This definition of an “HMW PEG” compound avoids the confusion in characterizing molecules such as the molecule of the preferred embodiment disclosed in the preceding paragraph, wherein an “HMW PEG” compound comprising at least two hydrocarbon chains and a hydrophobic core can be termed an HMW PEG compound or an HMW PEG derivative compound depending on one's perspective. As defined herein, such a molecule is an HMW PEG compound, regardless of whether it is regarded as derivatized or not. Exemplary functional groups include any of the alkoxy series, preferably C1-C10, any of the aryloxy series, phenyl and substituted phenyl groups. Such functional groups may be attached at any point to an HMW PEG molecule, including at either terminus or in the middle; also included are functional groups, e.g., phenyl and its substituents, that serve to link to smaller PEG molecules or derivative thereof into a single HMW PEG-like compound. Further, the HMW PEG-like molecules having an additional functional group may have one such group or more than one such group; each molecule may also have a mixture of additional functional groups, provided such molecules are useful in stabilizing at least one therapeutic during delivery thereof or in treating, ameliorating or preventing a disease, disorder or condition of an epithelial cell.

The following examples illustrate embodiments of the invention. Example 1 describes the protection against gut-derived sepsis provided to hepatectomized mice by high molecular weight PEG. Example 2 discloses how HMW PEG prevents pathogen adherence to intestinal epithelial cells. Example 3 reveals how HMW PEG inhibits pathogenic virulence expression generally, and PA-I lectin/adhesin expression specifically. Example 4 shows that PEG does not affect growth, or cell membrane integrity, of pathogens. Example 5 illustrates the unique topographical conformation of HMW PEG-coated pathogens using atomic force microscopy. Example 6 describes the cell-cell interactions affected by HMW PEG. Example 7 describes preventive methods using the compositions of the invention. Example 8 provides methods of protecting irradiated cells from radiation-induced damage and/or from microbial pathogens. Example 9 discloses methods for monitoring administration of HMW PEG, such as in the treatment methods of the invention, and corresponding kits.

### Example 1

### HMW PEG Protects Against Gut-Derived Sepsis Following 30% Hepatectomy

Male Balb/c mice were anesthetized and subjected to hepatectomy using a conventional protocol. A 30% bloodless excision of the liver along the floppy left lobe was performed. Control mice underwent manipulation of the liver without hepatectomy. The experimental and control groups each contained seven mice. In all mice, a volume of 200 μl of 10⁷ cfu/ml of Pseudomonas aeruginosa PA27853 was injected into the base of the cecum by direct needle puncture diluted in either saline, PEG 3.350 or PEG 15-20 (PEGs). The relatively low molecular weight PEGs are commercially available; PEG 15-20, having an average molecular weight of 15,000 to 20,000 daltons, is a combination of PEG 7-8 and PEG 8-10 covalently joined to a bis-phenol core. The PEG 7-8 has an average molecular weight of 7,000 to 8,000 daltons and the PEG 8-10 has an average molecular weight of 8,000 to 10,000 daltons. One of skill in the art will realize that HMW PEGs include compounds having any of a variety of PEG subunits with each subunit having any of a variety of average molecular weights joined, preferably covalently, to each other or to one or more linker molecules, which are relatively small molecules having functional groups suitable for joinder of PEG molecules. Suitable linkers substantially preserve the biological activity of HMW PEG (preservation of sufficient biological activity to realize a beneficial prophylactic or therapeutic effect as disclosed herein).

In order to provide a constant source of PEG for the 48-hour duration of the experiment, the needle was directed into the small bowel (ileum) and 1 ml of saline, PEG 3.35 or PEG 15-20 was injected retrograde into the proximal bowel. The puncture site was tied off with a silk suture and the cecum swabbed with alcohol. Mice were returned to their cages and were given H₂O only for the next 48 hours.

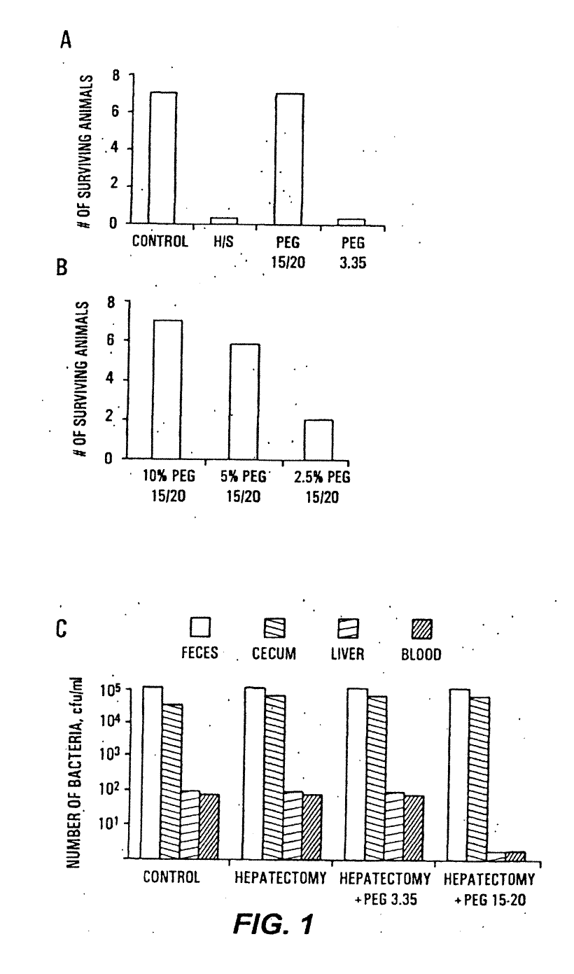

Dose response curves for PEG 15-20 are seen in panel b of FIG. 1. a. A statistically significant protective effect of PEG 15-20 was determined by the Fisher Exact Test (P<0.001). b. The minimum protective concentration of PEG 15-20 was determined to be 5% (P<0.05). c. Quantitative bacterial cultures of cecal contents (feces), washed cecal mucosa, liver, and blood 24 hours following 30% surgical hepatectomy and direct cecal injection of 1×10⁷ cfu/ml of PA27853. One-way ANOVA demonstrated a statistically significant increase in bacterial counts in cecal contents, mucosa, liver, and blood in mice following hepatectomy (P<0.001). A significant decrease (P<0.05) in the liver and blood bacterial counts was observed for PEG 3350, while PEG 15-20 completely prevented PA27853 from disseminating to the liver and blood of mice.

Pseudomonas aeruginosa strain ATCC 27853 (PA27853) is a non-mucoid clinical isolate from a blood culture. Direct cecal injection of strain PA27853 in mice previously subjected to a 30% bloodless surgical hepatectomy resulted in a state of clinical sepsis and no survivors at 48 hours. Mice subjected to sham laparotomy without hepatectomy (controls), who are similarly injected with P. aeruginosa, survive completely without any clinical signs of sepsis (FIG. 1a). To determine the ability of PEG solutions to prevent or lower mortality in this model, 200 μl of PA27853 at a concentration of 1×10⁷ cfu/ml, was suspended in one of two 10% (w/v) solutions of polyethylene glycol (PEG 3.35 versus PEG 15-20). PEG 3.35 was chosen as it represents the molecular weight of PEGs that have been available for clinical use for the last 25 years (Golytely®). In comparison, PEG solutions according to the invention that were used had molecular weights varying between 15-20 kDa. Suspended strains were introduced into the cecum by direct puncture. PEG 3.35 had no effect on mortality in mice following hepatectomy, whereas PEG 15-20 was completely protective. In fact, PEG 15-20 had a statistically significant protective effect, as determined by the Fisher Exact Test (P<0.001). Dose-response experiments demonstrated a 5% solution to be the minimal concentration of PEG 15-20 that was completely protective (P<0.05; see FIG. 1b), although one of skill in the art will recognize that HMW PEG solutions of less than 5% would be expected to provide some protection and, thus, fall within the scope of the present invention. With respect to bacterial counts in the experimental and control mice, a one-way analysis of variance (ANOVA) demonstrated a statistically significant increase in bacterial counts in the cecal contents, mucosa, liver, and blood in mice following hepatectomy (P<0.001). A significant decrease (P<0.05) in the liver and blood bacterial counts was observed for PEG 3.35, while PEG 15-20 completely prevented PA27853 from disseminating to the liver and blood of mice. PEG 15-20 completely inhibited the dissemination of intestinal PA27853 to the liver and bloodstream (FIG. 1c). The data indicate that the action of PEG solutions involves mechanisms that are non-microbiocidal. Given at PEG concentrations non-toxic to mammalian cells (i.e. ≦about 10%), no effect on bacterial growth patterns can be demonstrated.

The example demonstrates that HMW PEG reduces the mortality rate attributable to gut-derived sepsis in mice subjected to surgical intervention in the form of a partial hepatectomy. This mouse model indicates that HMW PEG therapy is useful in reducing the mortality rate of an animal species (i.e., reducing the likelihood of mortality in any given organism), such as a mammal, like man, subjected to a physiological stress such as invasive surgery (e.g., partial hepatectomy). It is expected that HMW PEG therapy will be effective in methods of preventing death or serious illness associated with sepsis when implemented following physiological stress (e.g., during post-operative care). Further, HMW PEG therapy may be used prior to physiological stressing (e.g., pre-operative care), under circumstances where introduction of the stress is predictable, to lower the risk of serious illness or death. HMW PEG therapy is also useful in ameliorating a symptom associated with a disease or abnormal condition associated with gut-derived sepsis.

### Example 2

### HMW PEG Prevents Pathogen Adherence to Intestinal Epithelia

Tight junctions are dynamic elements of the epithelial cell cytoskeleton that play a key role in the barrier function of the mammalian intestinal tract. P. aeruginosa results in a profound alteration in tight junctional permeability as measured by the transepithelial electrical resistance (TEER) of both Caco-2 cells and T-84 cells. Caco-2 cells are well-characterized human colon epithelial cells that maintain a stable TEER in culture, and this cell line provides a recognized in vitro model of the in vivo behavior of intestinal pathogens. To determine the protective effect of PEG on P. aeruginosa PA27853-induced decreases in TEER of cultured Caco-2 monolayers, 1×10⁷ cfu/ml of PA27853 was apically inoculated onto two Caco-2 cell monolayers in the presence of 10% PEG 3.35 or 10% PEG 15-20. TEER was serially measured for 8 hours and the maximal fall in TEER recorded.

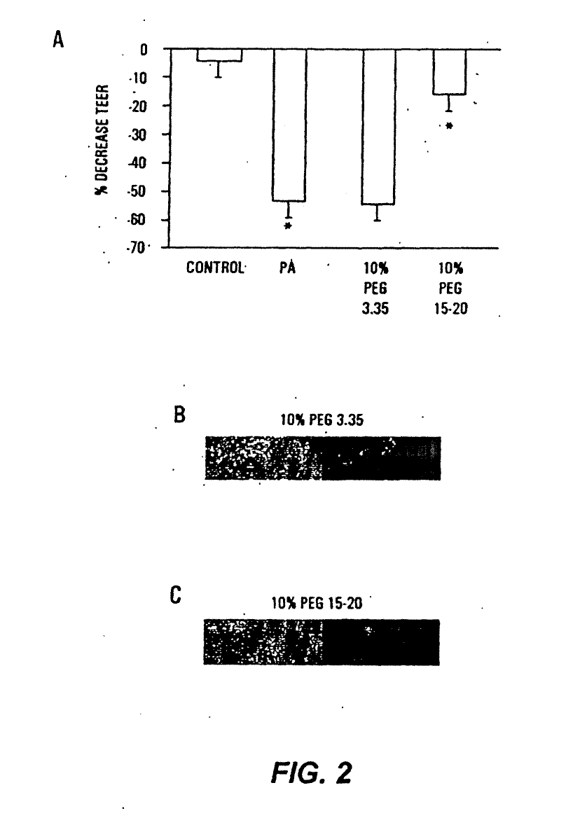

Only PEG 15-20 protected significantly against the P. aeruginosa-induced decrease in TEER (FIG. 2a). The data presented in FIG. 2 represent the mean±SEM % maximal fall in TEER from baseline of triplicate cultures (n=7) observed during 8 hours of apical exposure to 1×10⁷ cfu/ml of PA27853. A statistically significant decrease in TEER, as demonstrated in Caco-2 cells exposed to PA27853, was revealed by one-way ANOVA (P<0.001). A statistically significant protective effect on the fall in TEER induced by PA27853 was demonstrated for PEG 15-20 (P<0.001). FIG. 2b shows Caco-2 cells in the presence of PEG 3.35 and with apical exposure to PA27853. After 4 hours of co-culture in the presence of PEG 3.35, disruption of the Caco-2 cell monolayers displaying focally adherent bacteria was observed, with cells floating 30-40 microns above the monolayer scaffolds (FIG. 2b). In contrast, FIG. 2c, showing images of Caco-2 cells apically exposed for 4 hours to PA27853 in the presence of PEG 15-20, shows no evidence of floating cells in any of the planes examined. The protective effect of PEG 15-20 on Caco-2 cell integrity was associated with decreased bacterial adherence, reflected by a 15-fold higher recovery of bacteria in the cell supernatants following a 4-hour exposure to 1×10⁶ cfu/ml of PA27853.

The resistance of PEG-cultured human intestinal epithelial cells to the barrier-disrupting effects of P. aeruginosa, as judged by the maintenance of TEER, offers a practical approach to stabilizing tight junctional barrier function in the face of a challenge from invading pathogens. Further evidence of the therapeutic value of PEG 15-20 is that epithelial transport function (Na⁺/H⁺ exchange, glucose transport) is unaffected by this compound.

Thus, HMW PEG is relatively inert to, and has a stabilizing effect on, the intestinal epithelial barrier. The invention comprehends methods of treating intestinal barrier abnormalities associated with intestinal pathogens such as P. aeruginosa by administering HMW PEG to an animal such as a mammal and, preferably, a human. An intestinal barrier abnormality may be revealed by any diagnostic technique, or other means, known in the art. It is not necessary to identify an intestinal barrier abnormality prior to HMW PEG treatment, however. The low cost and high degree of safety associated with HMW PEG treatment make this approach suitable for both prophylactic applications, preferably directed towards at-risk organisms, as well as treatment methods applied to animals exhibiting at least one symptom characteristic of an intestinal barrier abnormality. The HMW PEG treatment methods would ameliorate a symptom associated with an intestinal barrier abnormality; preferably, the methods would reduce or eliminate the effects of gut-derived sepsis from a treated organism.

### Example 3

### HMW PEG Inhibits Virulence Expression in Pathogens

The expression of the PA-I lectin/adhesin in P. aeruginosa PA27853 was increased in the cecum of mice following hepatectomy and played a key role in the lethal effect of P. aeruginosa in the mouse intestine. PA-I functions as a significant virulence determinant in the mouse intestine by facilitating the adherence of PA27853 to the epithelium as well as by creating a significant barrier defect to the cytotoxins, exotoxin A and elastase. PA-I expression in P. aeruginosa is regulated by the transcriptional regulator RhlR and its cognate activator C4-HSL. Expression of PA-I in PA27853 was not only increased by exposure to C4-HSL, but also by contact with Caco-2 cells, Caco-2 cell membrane preparations, and supernatants from Caco-2 cell cultures.

Northern hybridization was used to analyze the expression of PA-I at the transcriptional level. Total RNA of P. aeruginosa was isolated by the modified three-detergent method. Probes were generated by PCR using PA-I primers: F(ACCCTGGACATTATTGGGTG) (SEQ ID NO: 1), R(CGATGTCATTACCATCGTCG) (SEQ ID NO: 2) and 16S primers: F(GGACGGGTGAGTAATGCCTA) (SEQ ID NO: 3), R(CGTAAGGGCCATGATGACTT) (SEQ ID NO: 4), and cloned into the pCR2.1 vector (Invitrogen, Inc.). The inserts were sequences that matched the sequence of either PA-1 or 16S. Specific cDNA probes for PA-1 and 16S were radiolabeled with α³²P-dCTP. The specific radioactivity was measured by a Storm 860 phosphorimager (Molecular Dynamics, CA), and relative percent changes compared to control were calculated based on the intensity ratio of PA-1 and 16S. Western blot was used for PA-I protein analysis, using rabbit affinity-purified polyclonal anti-PA-I antibodies. One ml of P. aeruginosa cells was washed with PBS and heated at 100° C. in lysis buffer (4% SDS, 50 mM Tris-HCl, pH 6.8); immunoblot analysis was performed by electrotransfer of proteins after Tricine SDS-PAGE. The PA-I lectin was detected by the ECL reagent (Amersham, N.J.).

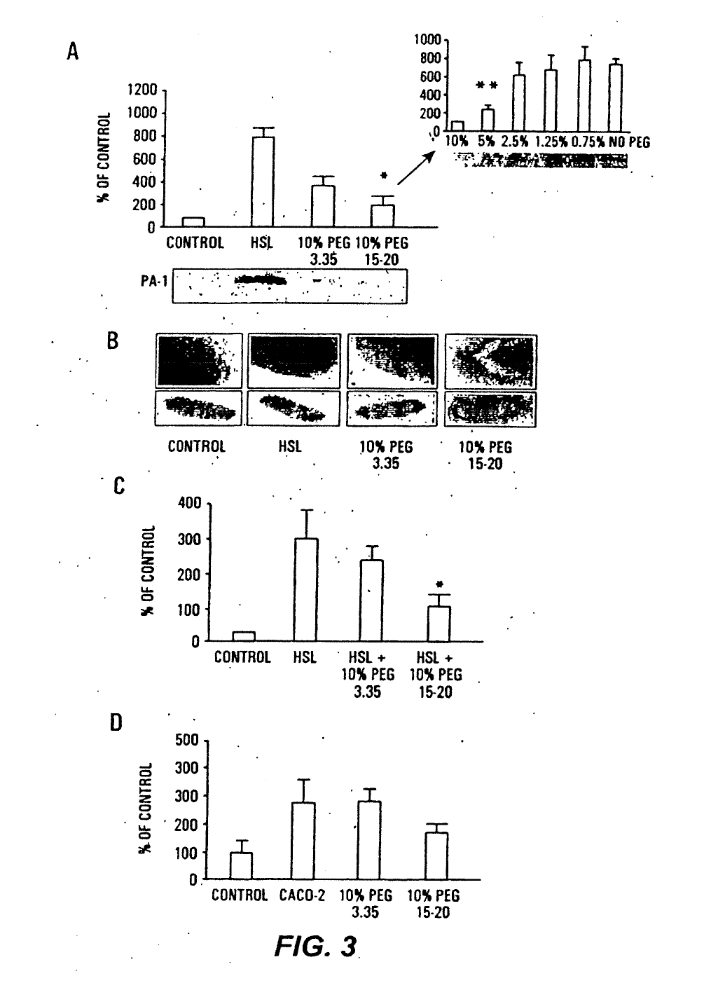

Exposure of P. aeruginosa PA27853 to 1 mM of the quorum-sensing signaling molecule C4-HSL resulted in a statistically significant increase (P<0.001, one-way ANOVA) in PA-I protein expression that was partially inhibited in the presence of 10% PEG 3.35 and inhibited to a much greater extent by 10% PEG 15-20 (FIG. 3). The minimum completely inhibitory concentration of PEG 15-20 on C4-HSL-induced PA-I expression was 5% (P<0.01, one-way ANOVA). Electron microscopic examination of individual bacterial cells exposed to C4-HSL in the presence and absence of PEG, demonstrated that C4-HSL caused a morphological change in the shape and pili expression of P. aeruginosa (FIG. 3b). The C4-HSL-induced morphological effect was completely eliminated in the presence of PEG 15-20, but not completely eliminated in the presence of PEG 3.35. A halo-type effect was seen surrounding PA27853 exposed to PEG 15-20 (FIG. 3b). For electron microscopy, PA27853 was inoculated in TSB with or without 1 mM C4-HSL and 10% HMW PEG and incubated overnight. One drop of 1% P. aeruginosa was stained with uranyl acetate and washed with 0.5M NaCl before examination under the electron microscope. Exposure of PA27853 to 0.1 mM of C4-HSL resulted in a statistically significant increase (P<0.001, one-way ANOVA) in PA-I mRNA expression assessed using Northern blots. The PA-I expression was greatly inhibited by 10% PEG 15-20. FIG. 3d shows that the increase in PA-I mRNA levels induced by a 4-hour exposure to Caco-2 cells was inhibited by PEG 15-20, but not by PEG 3.35 (P<0.001 one-way ANOVA).

The data presented herein show that a significant attenuation (3-4-fold decrease) of PA-I expression (protein and mRNA) in PA27853, induced by 100 μM-1 mM of C4-HSL, was observed when bacteria were pre-treated with 10% PEG 15-20 (FIG. 3a). Attenuation of C4-HSL-induced PA-I expression was also observed for 10% PEG 3.35, although the degree of attenuation was significantly less than that for 10% PEG 15-20. The minimum concentration of PEG 15-20 that inhibited C4-HSL induced expression of PA-I protein was 5% (FIG. 3b). Electron microscopy of individual bacterial cells exposed to C4-HSL demonstrated that C4-HSL caused a morphological change in the shape and pili expression of PA27853 (FIG. 3b). The C4-HSL-induced morphological effect was completely eliminated in the presence of PEG 15-20, but not PEG 3.35 (FIG. 3b). PA-I expression (mRNA), induced by 4 hours exposure to Caco-2 cells, was inhibited in the presence of PEG 15-20 but not PEG 3.35 (FIG. 3b). The protective effect of Caco-2 cell-induced PA-I expression with PEG 15-20 persisted in experiments of overnight exposure.

HMW PEG also affects the virulence expression of P. aeruginosa in response to known stimuli. The attenuation of C4-HSL-induced PA-I expression in PA27853 may be a major protective effect of PEG 15-20, given that quorum-sensing signaling is a well-established mechanism of virulence expression for this pathogen. The PEG 15-20-induced interference with Caco-2 cell-induced expression of PA-I is expected to be an important aspect of the protective effect of PEG 15-20. PEG 15-20 was found to have a protective effect on host animals through the attenuation of P. aeruginosa (PA27853) PA-I expression in response to filtered cecal contents (feces) from mice following 30% hepatectomy. The ability of PEG 15-20 to shield P. aeruginosa from host factors that increase its virulence expression is expected to be yet another mechanism by which organisms are protected from gut-derived sepsis.

Accordingly, the invention includes materials in the form of kits and corresponding methods of administering an HMW PEG to an animal to prevent or treat a condition characterized by the expression of a virulence factor or determinant by an intestinal pathogen such as one of the Pseudomonads. A virulence determinant may contribute to virulence directly, or indirectly. An example of an indirect contribution is the effect of the PA-I lectin/adhesin of P. aeruginosa on intestinal pathogen adhesion to intestinal epithelia and/or the generation of a barrier defect to the cytotoxins, exotoxin A and elastase.

### Example 4

### PEG does not Affect Cell Growth, or Cell Membrane Integrity, of Pathogens

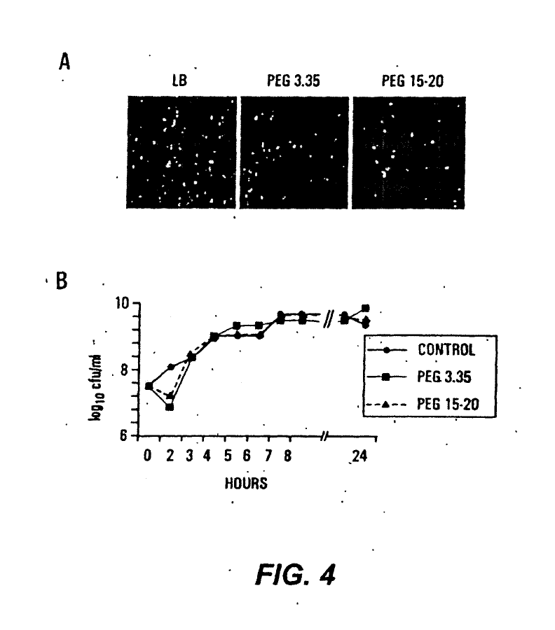

The effect of the two PEG solutions (PEG 3.35 and PEG 15-20) on bacterial membrane integrity was assessed by a staining method consisting of SYTO 9 and propidium iodide. Neither PEG solution had any effect on bacterial membrane permeability (FIG. 4a). Membrane integrity was determined using a live/dead bacterial viability kit (L-13152; Molecular Probes). Bacteria were quantified and counts expressed as cfu/ml by plating 10-fold dilutions of samples taken at different incubation times. Growth curves for P. aeruginosa grown overnight in TSB media containing either of the two PEG solutions demonstrated no inhibitory effect by either PEG solution on bacterial quantity (FIG. 4b). In fact, the growth pattern in each of the PEG-containing media was indistinguishable from the growth pattern in PEG-free TSB medium. The activity of a housekeeping enzyme involved in energy metabolism, lactate dehydrogenase (LDH), was measured at various time points during the exponential and stationary phases of growth. LDH activity was measured in a coupled diaphorase enzymatic assay using a substrate mix from CytoTox 96 (Promega). Protein concentration was determined using the BCA Protein Assay (Pierce). No change in LDH activity in cell-free supernatants of P. aeruginosa grown in the presence of PEGs was observed. The results of this experiment indicate that HMW PEG has a negligible effect on bacterial growth patterns.

The methods of the invention, and corresponding products (e.g., kits), provide the benefit of preventing or treating diseases or abnormal conditions associated with gut-derived sepsis without significantly influencing the composition of the intestinal flora. Similarly, the methods and products of the invention may be used to ameliorate a symptom associated with such diseases or abnormal conditions without significant change to the microbial composition of the intestine. One of skill in the art recognizes that methods (and kits) that do not significantly disturb the composition of the intestinal flora are desirable insofar as such methods would not be expected to lead to secondary health complications arising from such a disturbance.

### Example 5

### Atomic Force Microscopy of PEG-Coated Pathogen

One percent aliquots of a culture of PA27853 grown overnight were subcultured in tryptic soy broth (TSB), with or without 10% HMW PEG, for 4 hours at 37° C. One drop of each subculture was withdrawn and the P. aeruginosa PA27853 cells were extensively washed with PBS, dried on top of mica in blowing air for 10 minutes, and imaged immediately. Imaging of the dried bacteria with tapping-mode AFM was performed in air with a Multimode Nanoscope IIIA Scanning Probe Microscope (MMAFM, Digital Instruments). Subconfluent Caco-2 cells were treated with 10% HMW PEG for 4 hours and washed with PBS extensively. AFM imaging of the cells was performed in PBS without using an O-ring.

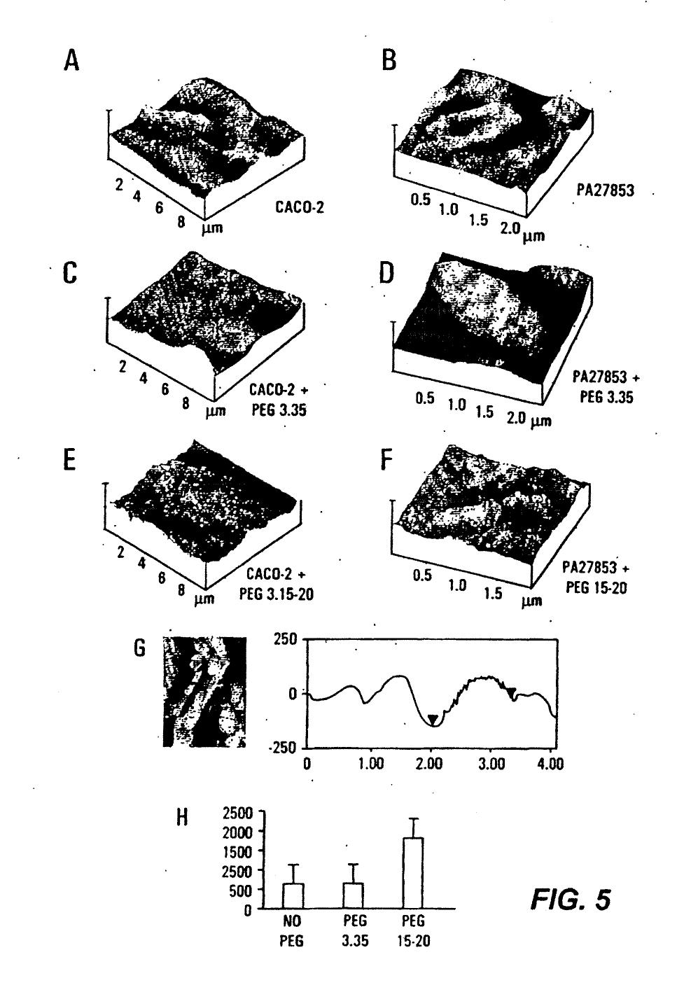

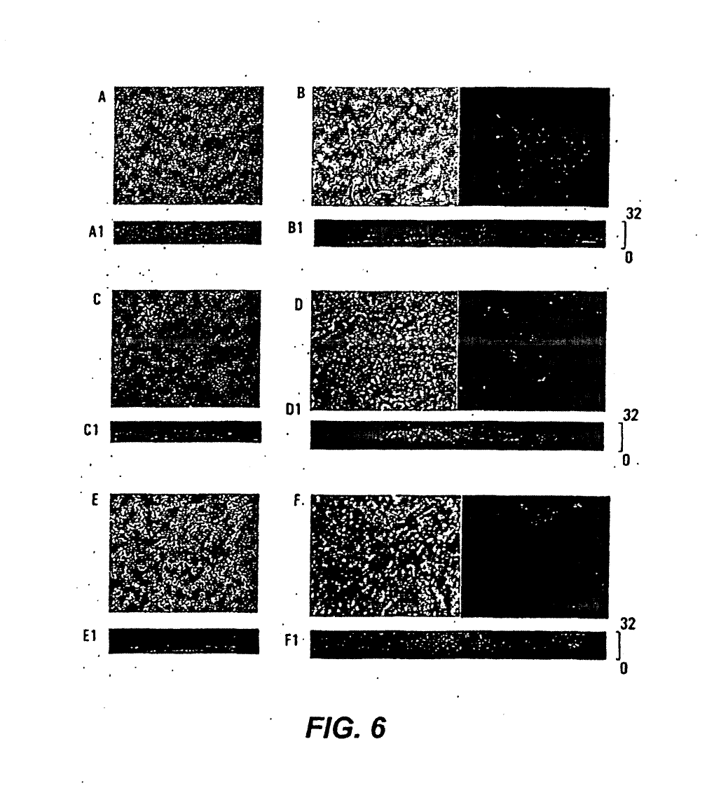

Atomic force microscopy of Caco-2 cells demonstrated a classical non-uniform surface with brush border microvili, while Caco-2 cells exposed to PEG 3.35 demonstrated a smooth planar appearance on the surface of the epithelial cells (FIGS. 5a, c). PEG 15-20 appears to carpet the Caco-2 cells by filling the asymmetries along a topographically defined plane (FIG. 5e), yielding a more complex topographically defined covering. In somewhat similar fashion, PA27853 cells exposed to PEG 3.35 demonstrate a pattern of smooth coating of the polymer to bacterial cells in a diffuse flat pattern (FIG. 6d), whereas PEG 15-20 appears to surround and hug the bacteria circumferentially in a more topographically asymmetric fashion. Cross-sectional analysis of the atomic force measurement of the bacterial diameter in PEG 15-20 demonstrates a significant increase in the bacteria/PEG envelope within the PEG solution (FIG. 5e, f). In other words, PEG 3.35 forms a smooth envelope around individual bacterial cells (FIG. 5e), whereas PEG 15-20 tightly hugs individual cells (FIG. 5f) and increases the polymer/bacterial diameter (FIGS. 5g, 5h), thereby distancing individual bacterial cells from each other.

Without wishing to be bound by theory, HMW PEG may exert its beneficial effect by the mere physical distancing of P. aeruginosa away from the intestinal epithelium. Alternatively, HMW PEG may provide benefits by preventing formation of a pathogenic quorum-sensing activation signal arising from cell-cell interaction of the pathogenic cells. Again without wishing to be bound by theory, it is possible that the coating of biological surfaces with HMW PEG results in loss of conformational freedom of the coating PEG chains and the repelling of approaching proteins. Polar-polar interactions between HMW PEG and Caco-2 cells could affect the elasticity of the PEG chains, constraining certain HMW PEG side chains to a molecular construct which repels protein. Data presented herein support the conclusion that HMW PEG-coated Caco-2 cells are more repellant to P. aeruginosa than uncoated Caco-2 cells, perhaps owing to a loss of “conformational entropy” as a result of some dynamic interaction of HMW PEG with Caco-2 cells.

The results of this experiment establish that HMW PEG treatment has an effect on treated cells, notably affecting the surface topology of such cells. Moreover, the effect of HMW PEG exposure on such cells is different from the effect that PEG 3.35 has on such cells. Although not wishing to be bound by theory, the results disclosed herein do provide a physical correlate for the markedly different effect on cells exhibited by HMW PEG relative to lower molecular weight PEGs, such as PEG 3.35.

### Example 6

### HMW PEG Affects Cell-Cell Interactions

To directly observe the effect of PEG solutions on the spatial orientation of P. aeruginosa, experiments were performed with live strains of P. aeruginosa PA27853/EGFP harboring the egfp gene encoding the enhanced green fluorescent protein. Experiments were performed in the presence and absence of Caco-2 cells. In order to image the effect of PEGs on both the bacteria and their interaction with the cultured epithelia, differential interference contrast (DIC) microscopy and GFP imaging were used.

The egfp gene encoding enhanced green fluorescent protein was amplified using the pBI-EGFP plasmid (Clontech) as a template. XbaI and PstI restriction sites were introduced using primers TCTAGAACTAGTGGATCCCCGCGGATG (SEQ ID NO: 5) and GCAGACTAGGTCGACAAGCTTGATATC (SEQ ID NO: 6). The PCR product was cloned directly into the pCR 2.1 vector using a TA-cloning kit (Invitrogen), followed by transformation of the pCR2.1/EGFP construct into E. coli DH5a. The egfp gene was excised from this construct by digestion with XbaI and PstI and the fragment containing the excised gene was cloned into the E. coli-P. aeruginosa shuttle vector pUCP24, which had been digested with the same restriction enzymes. The resulting construct (i.e., pUCP24/EGFP), containing the egfp gene in the shuttle vector, was electroporated at 25 μF and 2500 V into PA27583 electrocompetent cells. PA27853/EGFP-containing cells were selected on LB-agar plates containing 100 μg/ml gentamicin (Gm).

Cells harboring PA27853/EGFP were grown overnight in LB containing 100 μg/ml Gm, and 1% of the culture was used to inoculate fresh LB containing 50 μg/ml Gm. After 3 hours of growth, Isopropyl-β-D-thiogalactopyrano side (IPTG) was added to a final concentration of 0.5 mM, and cultures were incubated for 2 additional hours. 100 μl of the bacterial culture was mixed with 1 ml of HDMEM media (Gibco BRL) buffered with HEPES and containing 10% fetal bovine serum (HDMEM HF) and 10% HMW PEG. One ml of bacterial suspension was poured into a 0.15 mm-thick dTC3 dish (Bioptech). Four-day-old Caco-2 cells (p10-p30) grown in 0.15 mm-thick dTC3 dishes (Bioptech) in HDMEM HF were washed once in HDMEM HF with or without HMW PEG. One ml of bacterial suspension prepared as above was added to a dTC3 dish containing Caco-2 cells. The dispersion pattern of bacterial cells in dTC3 dishes was observed directly with an Axiovert 100 TV fluorescence inverted microscope using DIC and GFP fluorescence filters, at an objective magnification of 63×. The temperature was adjusted with a Bioptechs thermostat temperature control system. Tungsten lamps (100 V) were used for both DIC and the GFP excitation. The 3D imaging software (Slidebook) from Intelligent Imaging Innovations was used to image the bacterial cell dispersion pattern in the Z plane using the GFP filter. Uniformly dispersed planktonic P. aeruginosa cells in the medium without Caco-2 cells were seen on a DIC image (FIG. 6a₁) and Z plane reconstruction (FIG. 6a₂). In the presence of Caco-2 cells, bacterial cells developed a clumped appearance (FIG. 6b₁) and were seen adhering to the Caco-2 cells (FIG. 6b₂). A solution of 10% PEG 3.35 decreased the bacterial motility and induced immediate formation of mushroom-shaped bacterial microcolonies (FIG. 6c₁) adhering to the bottom of the well (FIG. 6c₂). In the presence of Caco-2 cells, bacterial microcolonies were approximately 8 microns above the plane of the epithelial cells (FIG. 6d_{1,2}). A solution of 10% PEG 15-20 greatly diminished the motility of P. aeruginosa cells. Nevertheless, for the first 0.5-1 hour of incubation in PEG 15-20-containing medium, bacterial cells formed spider leg-shaped microcolonies that were close to the bottom of the well (FIG. 6e_{1,2}). Within several hours, spider leg-shaped microcolonies occupied the entire space/volume of the medium. In the presence of Caco-2 cells, P. aeruginosa cells lost the spider leg-like configuration and were seen elevated high above the plane of the epithelium (30-40 microns) (FIG. 6f_{1,2}).

To determine the spatial orientation of the bacterial-epithelial cell interactions in three dimensions, Z plane re-constructions were performed. Images demonstrated that the two PEG solutions had different effects on the clumping behavior of P. aeruginosa and differentially affected the spatial orientation of the bacteria depending on the presence or absence of Caco-2 cells. In experiments with medium only, P. aeruginosa were seen to display a uniformly dispersed pattern (FIG. 6a). Bacterial cells examined in the presence of Caco-2 cells, however, developed a clumped appearance and were seen adjacent to the plane of the epithelial cells at the bottom of the wells (FIG. 6b). Bacterial cells examined in the presence of PEG 3.35 alone formed large clumped aggregates and remained in the bottom of the culture well (FIG. 6c), whereas bacterial cells examined with Caco-2 cells in medium containing PEG 3.35, remained suspended above the plane of the epithelial cells (about 8 microns), maintaining their clumped appearance (FIG. 6d). Bacterial cells examined in the presence of PEG 15-20 alone displayed a uniform pattern of microclumping (FIG. 6e), whereas bacterial cells examined in the presence of Caco-2 in medium containing PEG 15-20 were suspended higher above the plane of the epithelium (˜32 microns) in clumped formation (FIG. 6f). In timed experiments, bacterial motility was observed to be decreased by PEG 3.35 and, to an even greater degree, with PEG 15-20.

In a manner analogous to the experiment disclosed in Example 5, this Example provides a physical correlate for the observed effect of HMW PEG on cell-cell interaction, consistent with its beneficial prophylactic and therapeutic activities as disclosed herein. It is expected that use of HMW PEG will reduce or eliminate deleterious cell-cell interactions in the intestine (e.g., between intestinal epithelial cells and intestinal pathogens such as the Pseudomonads), reducing the risk of diseases and/or abnormal conditions associated with gut-derived sepsis.

### Example 7

### Methods of Preventing Disease/Abnormal Conditions

The invention also provides methods of preventing a variety of diseases and/or abnormal conditions in humans and other animals, particularly other mammals. In these methods, an effective amount of HMW PEG is administered to a human patient or an animal subject in need thereof. The PEG may be administered using a schedule of administration that is determined using routine optimization procedures known in the art. Preferably, the PEG has an average molecular weight of 5,000-20,000 daltons, and more preferably between 10,000-20,000 daltons. It is contemplated that at least 5% HMW PEG is administered. The HMW PEG may be administered in any suitable form, e.g., as a solution, as a gel or cream, as a solution suitable for nebulizing (e.g., for inhalational use), in a pharmaceutical composition comprising the HMW PEG, and in a sterile, isotonic solution suitable for injection into an animal. Administration may be accomplished using any conventional route; it is particularly contemplated that the HMW PEG is administered orally or topically. In some embodiments, the HMW PEG composition being administered further comprises a compound selected from the group consisting of dextran-coated L-glutamine, dextran-coated inulin, dextran-coated butyric acid, a fructo-oligosaccharide, N-acetyl-D-galactosamine, dextran-coated mannose, galactose and lactulose. In another embodiment, the administered HMW PEG composition further comprises dextran-coated L-glutamine, dextran-coated inulin, dextran-coated butyric acid, one or more fructo-oligosaccharides, N-acetyl-D-galactosamine, dextran-coated mannose, galactose and lactulose.

The invention provides methods of preventing a variety of diseases and abnormal conditions, such as swimmer's ear, acute or chronic otitis media, ventilator-associated pneumonia, gut-derived sepsis, necrotizing enterocolitis, antibiotic-induced diarrhea, pseudomembranous colitis, inflammatory bowel diseases, irritable bowel disease, neutropenic enterocolitis, pancreatitis, chronic fatigue syndrome, dysbiosis syndrome, microscopic colitis, chronic urinary tract infection, sexually transmitted disease, and infection (e.g., exposure to an environment contaminated by a bioterror agent such as Bacillus anthracis, Small Pox Virus, enteropathogenic E. coli (EPEC), enterohemorrhagic E. coli (EHEC), enteroaggregative E. coli, (EAEC), Clostridium difficile, rotavirus, Pseudomonas aeruginosa, Serratia marcescens, Klebsiella oxytocia, Enterobacteria cloacae, Candida albicans, Candida globrata, and the like). In a preferred embodiment of the method of preventing chronic urinary tract infection, or treating such an infection, the HMW PEG is delivered in the form of a bladder irrigant. For sexually transmitted disease prevention, a composition of the invention is preferably used to lubricate a condom. In a preferred embodiment of a method of preventing infection by a bioterror agent, the composition according to the invention is provided in the form of a gel or cream, suitable for topical application. It is expected that such topical application will be useful in preventing a variety of diseases/abnormal conditions associated with any of the bioterror agents or associated with a variety of chemical or physico-chemical agents that pose a threat to man or animal in terms of survival, health or comfort. Such chemical or physico-chemical agents include those agents capable of burning or otherwise injuring skin and which are rendered inactive or are poorly soluble in the compositions of the invention.

In one embodiment of the preventive methods, male Balb/c mice are anesthetized and an aqueous 5% solution of PEG 15-20 is injected into the base of the cecum by direct needle puncture. In order to provide a constant source of PEG for the 48-hour duration of the experiment, the needle is directed into the small bowel (ileum) and 1 ml of the PEG 15-20 is injected retrograde into the proximal bowel. The puncture site is tied off with a silk suture and the cecum swabbed with alcohol. Mice are returned to their cages and are given H₂O only. Forty-eight hours later, the mice are subjected to a conventional hepatectomy procedure involving a 30% bloodless excision of the liver along the floppy left lobe. Control mice will experience manipulation of the liver without hepatectomy. The preventive treatment involving administration of HMW PEG is expected to reduce or eliminate the incidence of surgery-associated gut-derived sepsis in mice.

These methods are applicable beyond the preventive care of such pets as mice, guinea pigs, dogs and cats to such agriculturally significant animals as cattle, horses, goats, sheep, pigs, chickens, turkeys, ducks, geese, and any other domesticated animal. Moreover, these preventive methods are expected to be applicable to humans, improving the health, and life expectancy, of many patients or candidates at risk of developing a disease and/or an abnormal condition, such as swimmer's ear, acute or chronic otitis media, ventilator-associated pneumonia, gut-derived sepsis, necrotizing enterocolitis, antibiotic-induced diarrhea, pseudomembranous colitis, an inflammatory bowel disease, irritable bowel disease, neutropenic enterocolitis, pancreatitis, chronic fatigue syndrome, dysbiosis syndrome, microscopic colitis, chronic urinary tract infections, sexually transmitted diseases, and infectious agents (e.g., bioterror compositions) that include, but are not limited to, anthrax and small pox. As noted above, the preventive methods comprise administration of a composition comprising at least 5% HMW PEG (5-20 kDa), by any known or conventional administration route, to man or another animal. Preferably, the preventive methods are practiced on those individuals at risk of developing one or more of the aforementioned diseases and/or abnormal conditions, but it is contemplated that the compositions and methods of the invention will be useful in either a prophylactic or therapeutic role to broadly treat or prevent such diseases or abnormal conditions in entire populations or sub-populations of man or other animals.

### Example 8

### Methods of Protecting Irradiated Cells from Radiation-Induced Damage and/or from Microbial Pathogens

This example provides experimental results establishing the ability of HMW PEG 15-20 to protect intestinal epithelial cells and mice from radiation-injury complicated by the presence of P. aeruginosa. More particularly, this example provides data establishing that HMW PEG and like compounds prevent epithelial cell apoptosis, shield against bacterial invasion, and protect mammals from mortality following radiation and exposure to a microbial pathogen such as P. aeruginosa. Polyethylene glycols and oxides are known to shield solid surfaces from bacterial adhesion, thereby preventing biofilm formation. Further, it is known that a non-absorbed HMW PEG co-polymer is protective against adhesion of P. aeruginosa to intestinal epithelial cells and reduces mortality in mice following the mechanical stress of surgery.

Exposure of cells to radiation, however, does not mechanically stress cells in a manner analogous to surgical intervention. Published reports have established that relatively low molecular weight PEG (PEG 200-600), when injected, provided some protection against irradiation of the head and neck (U.S. Pat. No. 4,676,979). This group also reported that higher molecular weight forms of PEG (e.g., PEG 1,000, PEG 1,450, PEG 4,000 and PEG 20,000) were dose-limited by toxicity and had to be diluted to yield an injectable composition, resulting in no protection against systemic irradiation (Schaeffer et al., Rad. Res. 107:125-135 (1986)).

In view of the art, it was surprising that HMW PEG was found to protect eukaryotic cells, such as mammalian epithelial cells, from the deleterious effects of microbial pathogens (e.g., P. aeruginosa), even when those mammalian cells had been rendered vulnerable to such effects by exposure to radiation. The results establish that HMW PEG provided a protective effect to radiated intestinal epithelial cells and abdominally radiated mice exposed to P. aeruginosa.

In general, the experiments described in this Example involved materials and methods consistent with the materials and methods disclosed and described throughout the document. To avoid any confusion, however, certain materials and methods used in the experiments described in this Example are provided immediately below, followed by a disclosure of the experimental results and a discussion of the significance of those results.

Bacterial Strains. P. aeruginosa strains PAO1 and its derivative, strain PAO1/EGFP harboring the egfp gene encoding the green fluorescent protein (EGFP) on the pUCP24 plasmid vector that is expressed under control of the Plac promoter, as well as the PA-I lectin/adhesin luminescent reporter strain PAO1/lecA::lux, were used.

Epithelial Cell Lines. Rat intestinal epithelial cells (IEC-18 cells, passage 30-34) were used in all experiments. IEC-18 cells were originally derived from the ileum of rat intestine and display growth characteristics that are observed with normal intestinal epithelial cells. Under in vitro conditions, the cells exhibit contact-inhibited cell growth, do not form colonies in soft agar, and are not tumorigenic when injected in nude mice. IEC-18 cells were routinely cultured in plastic culture flasks containing DMEM supplemented with 10% fetal calf serum, 10 mM glutamine, 50 U/ml penicillin, and 50 μg/ml streptomycin at 37° C. in a humidified atmosphere of 5% CO₂-95% air. Cells were subcultured weekly using 0.05% trypsin-0.02% EDTA in phosphate-buffered saline (PBS) without Ca²⁺ and Mg²⁺.

High Molecular Weight Polyethylene Glycol (PEG 15-20). High molecular weight polyethylene glycol 15,000-20,000 Da (catalog no. P2263), herein referred to as PEG 15-20, was purchased from Sigma (St. Louis, Mo.). For imaging studies, unmodified PEG 15-20 was fused with fluorescein as custom-prepared by Sigma (batch 478-004-2, 28 Nov. 06) and visualized using the Xenogen in vivo imaging system.

In vivo Imaging of Ingested Fluorescein-labeled PEG 15-20. In order to assess the distribution of PEG in vivo in the gastrointestinal tract, groups of 8-week-old male C57BL6 mice (n=3/group) were provided with sterile drinking water supplemented for one week with fluorescein-labeled PEG 15-20 (1% solution) (IACUC protocol #71744). To enhance palatability of the PEG 15-20 solution, 5% dextrose was added. Control mice were given 5% dextrose in sterile water only. All mice were allowed a normal chow diet during the study period. Following one week of ad libitum drinking, the 1% PEG 15-20 solution was discontinued and all mice were allowed to drink 5% dextrose in water only. The Xenogen IVIS 200 in vivo fluorescence imaging system (Optical Imaging Core Facility, University of Chicago) was used to capture daily sequential abdominal images of mice beginning from the day of discontinuation of the PEG 15-20 solution until a signal was no longer obtainable. Prior to imaging, all mice were anesthetized with intra-peritoneal injection of ketamine/xylazine and their hair removed with Nair solution to enhance signal sensitivity. For cecum images, mice were sacrificed, and distal intestines were removed and the lumen vigorously irrigated with saline (flushed 10× with 5 ml). Images were taken using an SZX16 Olympus stereomicroscope.

Atomic Force Microscopy. IEC-18 cells were grown to a confluent monolayer in IEC growth media and then treated with a 10% PEG 15-20 solution for four hours. Cells were then extensively washed with additional growth media and imaged with tapping mode atomic force microscopy (AFM) performed in air with a Multimode Nanoscope IIIA Scanning Probe Microscope without using an O-ring (MMAFM; Digital Instruments, Woodbury, N.Y.). Intestinal tissue sections were harvested from mice (n=3/group) that had been provided a 5% PEG 15-20 solution or tap water to drink ad libitum, along with a normal chow diet, for one week prior to sacrifice. Harvested intestinal sections included three mm samples from the terminal ileum and proximal colon. Sections were opened longitudinally with sharp dissection, placed luminal side up on mica, and imaged with tapping mode AFM.

Exposure of Cultured Intestinal Epithelial Cells to Radiation. IEC-18 cells were either grown to 90% confluency in 24-well plates (for subsequent apoptosis assays) or in glass-bottom culture dishes to a confluent monolayer (MatTek, Ashland, MA-01721 Part No. P35GCol-0-14-C collagen coated). Three groups of cells were examined: Group I served as a control group and contained cells incubated in standard IEC growth media for 24 hours; Group II (5 Gy) included cells exposed to 5 Gy of irradiation (generated from a Co-60 gamma source at a dose rate of 1.27 Gy/minute) with subsequent growth for 24 hours; Group III (5% PEG+5 Gy) had cells apically exposed to 5% PEG 15-20 in IEC growth media for 1 hour, followed by gentle washing 3 times with growth media, and irradiation with 5 Gy, with subsequent growth for 24 hours. Growth media was changed before the irradiation in all groups.

Mouse Model of Post-Abdominal Radiation Gut-Derived Sepsis Induced by P. aeruginosa. The mouse model of radiation-induced gut-derived sepsis was developed using eight-week-old male C57BL6 mice. Under IACUC protocols 70931 & 71744, all mice (n=10/group) were fasted 12 hours prior to a single 13 Gy dose of abdominal irradiation delivered from an x-ray-generating irradiator. Following irradiation, all mice continued to fast but were allowed to drink ad libitum either a 5% dextrose solution in water (control) or 1% PEG 15-20 solution in water containing 5% dextrose for 48 hours. Mice then underwent surgical laparotomy under general anesthesia (IP injection of ketamine/xylazine) and were directly inoculated with 200 μL of 10⁷ CFU/ml of P. aeruginosa PAO1 in a 10% glycerol solution using a 27-gauge syringe via cecal puncture. Following cecal injection, the abdominal cavity was closed in two layers and mice were allowed to recover under a warming lamp. The direct cecal puncture model has been previously described and characterized and is a reliable model of lethal gut-derived sepsis. Post-operatively, both groups of mice were observed for mortality and maintained on their respective drinking solutions (5% dextrose in water vs. 1% PEG 15-20 in water supplemented with 5% dextrose) and transitioned to normal mouse chow diet 24 hours post-surgery and maintained under these conditions for the remainder of the experiment. All mice were followed and observed twice daily for the development of sepsis. When possible, mice were sacrificed and counted as a mortality when they were frankly septic (lethargy, chromodacryorrhea, liquid stools, ruffled fur) and frankly moribund. In experiments in which PEG 15-20 was evaluated for its effect on intestinal epithelial apoptosis and expression of p53 and p21, reiterative studies were performed in a limited number of mice (n=3) without laparotomy and injection of P. aeruginosa where mice were sacrificed 24 hours post radiation.

Cell Death and Apoptosis Assays. A cell death ELISA kit (Cell Death Detection ELISA^{PLUS}, Roche Molecular Biochemicals) was used to detect cytoplasmic histone-associated DNA fragments. Twenty-four hours after irradiation, IEC-18 cells were treated with lysis buffer (890 μl ddH₂0, 10 μl of 1M Tris-HCl, pH 8.0, 5 μl of 1M MgSO₄, 5 μl RNAse, 5 μl DNAse, 100 μl of 10× Complete Protease Inhibitor cocktail, Roche), and 20 μl of cell lysate was used for ELISA. Nuclear DNA fragmentation was detected using the TUNEL assay (In Situ Cell Death Detection Kit, POD (peroxidase), Roche Diagnostics—cat. no. 11684817910). Briefly, IEC growth media was discarded, cells were fixed in 4% formaldehyde, permeabilized in 0.2% Triton X-100 and incubated with TdT incubation buffer for 60 minutes in a 37° C. humidified incubator for 3′-OH labeling. Stained cells were analyzed using fluorescent confocal microscopy.

TUNEL assay and hemotoxylin&eosin (H&E) staining of intestinal epithelium in mice. Routine 5-μm paraffin sections of control, 5 Gy-irradiated or PEG-pre-treated 5 Gy-irradiated mouse ilea were prepared. The TUNEL assay was performed using the In Situ Cell Death Detection Kit, POD (Roche Applied Science).

Detection of the p53 and p21 expression. p53 expression and p21 expression in intestinal epithelial IEC-18 cells were determined by immunoblotting using anti-p53 antibodies (sc-99 mouse monoclonal antibody raised against amino acids 156-214 of p-53 of human origin, Santa Cruz Biotechnology) and anti-p21 antibody (Santa Cruz Biotechnology), where appropriate. IEC-18 cell monolayers were exposed to 5 Gy of irradiation and allowed to incubate for 24 hours in growth media with or without 5% PEG 15-20. Afterward, cells were collected and lysed using cell lysis buffer as described herein. The protein concentration in cell lysates was determined using BioRad Standard Protein Assay Reagent (Hercules, Calif.). Proteins in lysates (25 μg per well) were separated by 10% SDS-PAGE followed by transfer to a polyvinylidene difluoride (PVDF) membrane (Polyscreen; NEN, Boston, Mass.) in 1× Towbin buffer (25 mM Tris, 192 mM glycine [pH 8.8], 10% [vol/vol] methanol). All membranes were blocked in 5% (wt/vol) non-fat dry milk in Tween-Tris-buffered saline (T-TBS; 150 mM NaCl, 5 mM KCl, 10 mM Tris [pH 7.4], 0.05% [vol/vol] Tween 20). Blots were incubated overnight at 4° C. with anti-p53 or anti-p21 antibodies, as appropriate. Membranes were then washed five times with T-TBS, incubated with anti-mouse horseradish peroxidase-conjugated secondary antibody for 1 hour at room temperature, and then washed four times with T-TBS and once with Tris-buffered saline. SuperSignal West Pico Chemiluminescent Substrate (Pierce Chemical, Rockford, Ill.) system was used to visualize the bands of interest. The expression of p53 and p21 in mouse intestinal tissues was determined by immunohistochemistry. Mice drinking water versus a 1% PEG 15-20 solution in water (n=3/group) were sacrificed 24 hours following irradiation (without intestinal P. aeruginosa inoculation), and a total of ten 3-mm intestinal cross-sections were harvested immediately from the distal ileum and proximal colon of each mouse. Tissue samples were immediately placed in a 10% formalin solution and subsequently embedded in paraffin. Cross-sectional slides were stained with either H&E (Hematoxylin & Eosin), anti-p53 antibodies, or anti-p21 antibodies. The p53-to-nuclear staining ratio and p21-to-nuclear staining ratio were calculated using ACIS software (Automated Cellular Imaging Software) by a trained pathologist in a blinded fashion.

Spatial orientation of P. aeruginosa to IEC-18 cells. The ability of PEG to prevent bacterial adherence to radiated cells was assayed using IEC-18 cells grown in glass-bottom culture dishes (MatTek, Ashland, MA-01721 Part No. P35GCol-0-14-C collagen coated) apically exposed to P. aeruginosa producing green fluorescent protein (PAO1/EGFP). Briefly, 24 hours following radiation, 100 μL of P. aeruginosa strain PAO1/EGFP (10⁷ CFU/mL) were added to each glass-bottom culture dish containing 1 mL of growth media. The expression of the egfp gene from Plac was induced by 0.5 mM isopropylthiogalactoside. Real-time imaging was performed using laser scanning confocal microscopy (Leica, model TCS SP2 AOS) to image the Z-plane dispersion pattern using a GFP filter. The images created a series of sequential Z-stacks that allowed the assessment of time-dependent adhesion of GFP-tagged bacteria to IEC-18 cell monolayers. Z-stack data were analyzed using Image J software available from the National Institutes of Health.

Statistical Analysis. All statistical analyses of the data were performed using the Student's t-test and analysis of variance (ANOVA) with Microsoft Excel and Sigma Plot software. Kaplan-Meier Survival analysis was performed using SPSS 15.0 software.

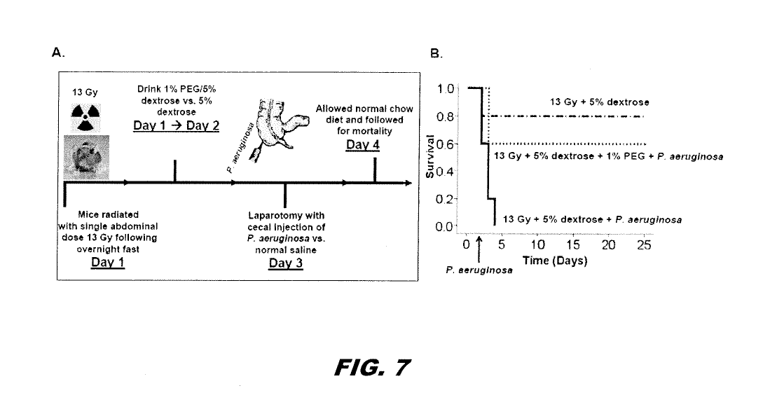

The results of these experiments established a number of facts, including confirmation that PEG 15-20 protects against radiation-induced gut-derived sepsis. In order to recapitulate the natural history of gut-derived sepsis following abdominal radiation, C57BL6 mice were abdominally radiated with 13 Gy and assigned to drink 5% dextrose (D5W) or D5W supplemented with PEG 15-20 as their exclusive water source. Forty-eight hours later, mice were directly inoculated in the intestine (cecal injection via laparotomy) with P. aeruginosa to mimic the clinical effects of abdominal radiation where there is loss of the protective probiotic flora and overgrowth of predominant gram-negative opportunistic pathogens. Mortality was followed for up to three weeks. As seen in FIG. 7B, survival curves demonstrated a major protective effect against mortality when mice drank PEG 15-20 compared to D5W alone (n=10, p<0.001). Control mice receiving 13 Gy abdominal irradiation only (no P. aeruginosa) demonstrated a 20% mortality on day 25; a result that did not significantly differ from irradiated mice receiving both P. aeruginosa and PEG 15-20 (n=10, p=NS).

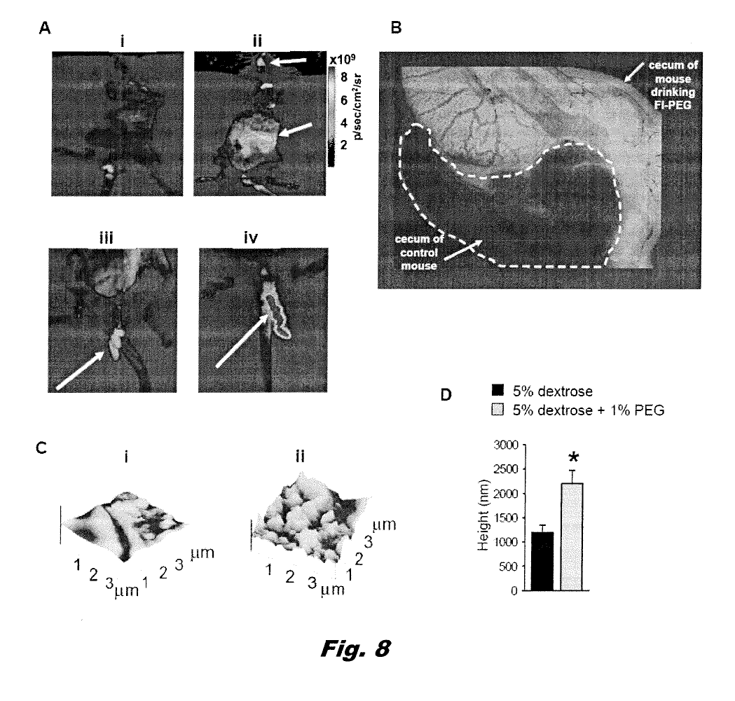

The data also establish that orally administered PEG 15-20 provides a protective effect in coating the luminal surface of the gastrointestinal tract. In order to determine the gastrointestinal distribution and retention of orally ingested PEG 15-20 in vivo, mice were allowed to drink a 1% solution of fluorescein-labeled PEG 15-20 (Fl-PEG) for seven days. The PEG solution was then discontinued and mice were allowed a normal diet and followed with daily in vivo fluorescence imaging (Xenogen). FIG. 8A depicts in vivo images of live mice having consumed a control solution containing 5% dextrose (FIG. 8A-i) or a 1% solution of Fl-PEG (FIG. 8A-ii).

Animals drinking 1% Fl-PEG displayed distribution of the polymer in multiple regions of the gastrointestinal tract, including the mouth (pharynx), midgut/hindgut, and within expelled feces. Ex vivo intestinal segments imaged by fluorescent microscopy (FIG. 8B), demonstrated durable retention of the fluorescence along the length of the intestinal tract for up to 7 day following its discontinuation. To determine whether the above findings represented the presence of PEG 15-20 on the epithelial surface, midgut segments of mouse intestine were harvested from mice drinking either D5W alone or D5W with PEG 15-20 and atomic force microscopy (AFM) of the wet segments was used to determine the height of deflection off the epithelial surface as a function of the presence of the polymer. Mice drinking HMW PEG (PEG 15-20) had a topographically distinct appearance to their mucosal surface and a significantly higher height of deflection measurement by AFM (FIG. 8C-D).

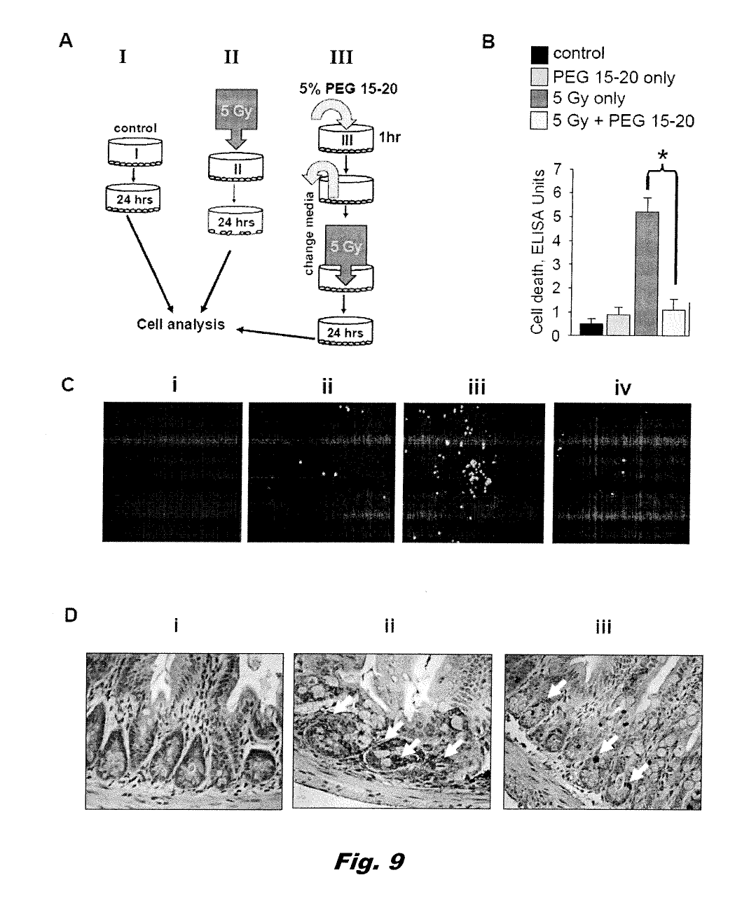

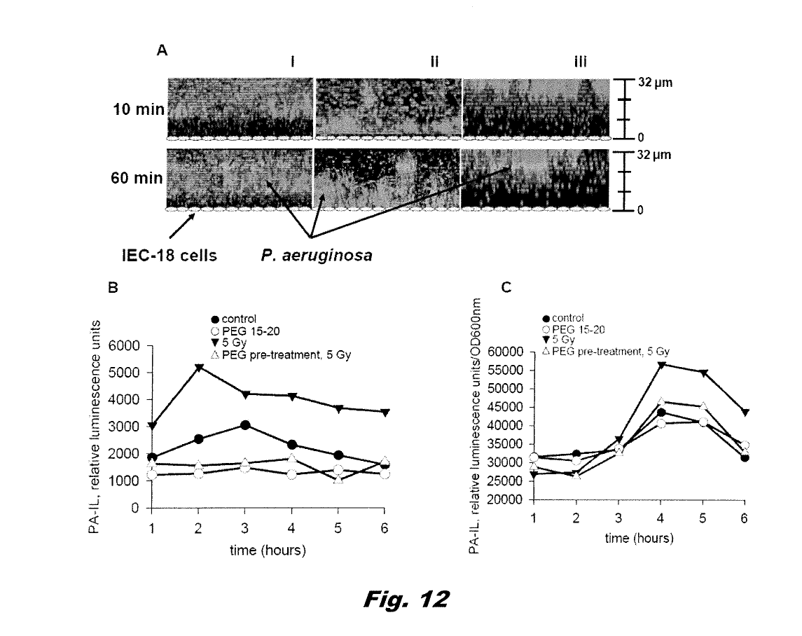

The data further show that PEG 15-20 affected epithelial cell apoptosis through pathways involving p53 and p21. To determine the direct effect of PEG 15-20 on epithelial cell apoptosis, IEC cells were apically exposed to HMW PEG for one minute or one hour, followed by exposure to 3-5 Gy radiation and then, at 24 hours, examined for cell death and apoptosis using standard TUNEL and ELISA cell death assays. Control cells were treated with medium alone, medium+irradiation, or medium+PEG without irradiation. An exemplary protocol is outlined in FIG. 9A. As might be expected, IEC cells exposed to 5 Gy of radiation formed cytoplasmic histone-associated DNA fragments (*p<0.001) as measured by ELISA (FIG. 9B), an effect that was markedly attenuated when IEC were pretreated with PEG 15-20. TUNEL assays confirmed these findings (FIG. 9C). TUNEL assays performed on mouse ileum obtained 24 hours following abdominal radiation (13 Gy) demonstrated a similar in vivo protective effect of PEG 15-20 (FIG. 9D). Radiation-induced epithelial cell apoptosis was reduced by 80% when exposed to HMW PEG versus control (FIG. 9). Irradiated IEC-18 cells displayed a high degree of invasion by P. aeruginosa adherence that was significantly decreased with HMW PEG (FIG. 12A). The presence of intestinal P. aeruginosa significantly altered the mortality of abdominal radiation exposure (FIG. 7). HMW PEG significantly protected mice against this effect (FIG. 7).

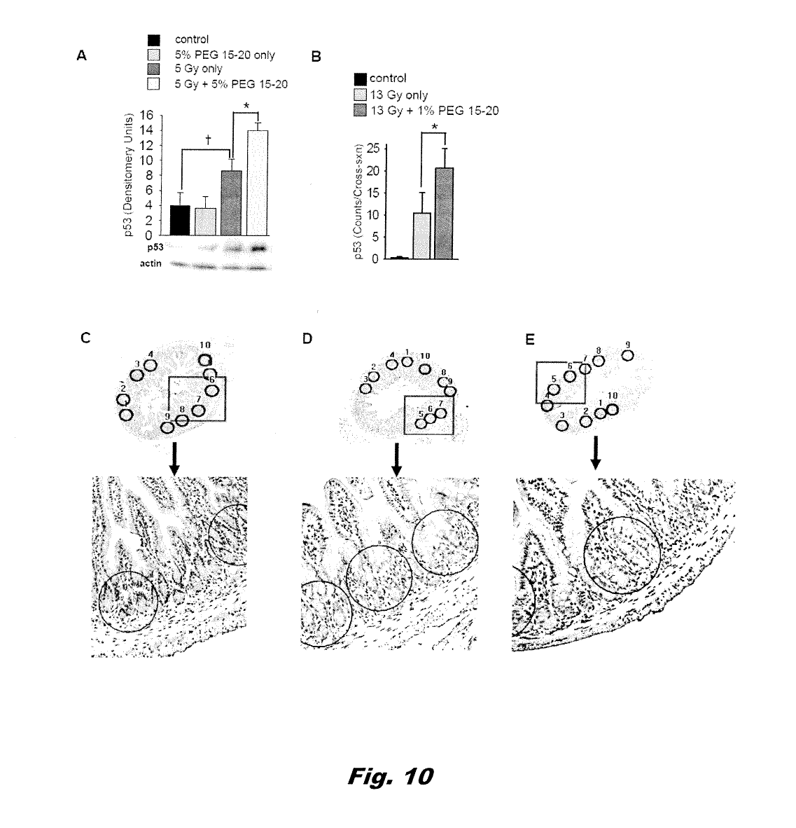

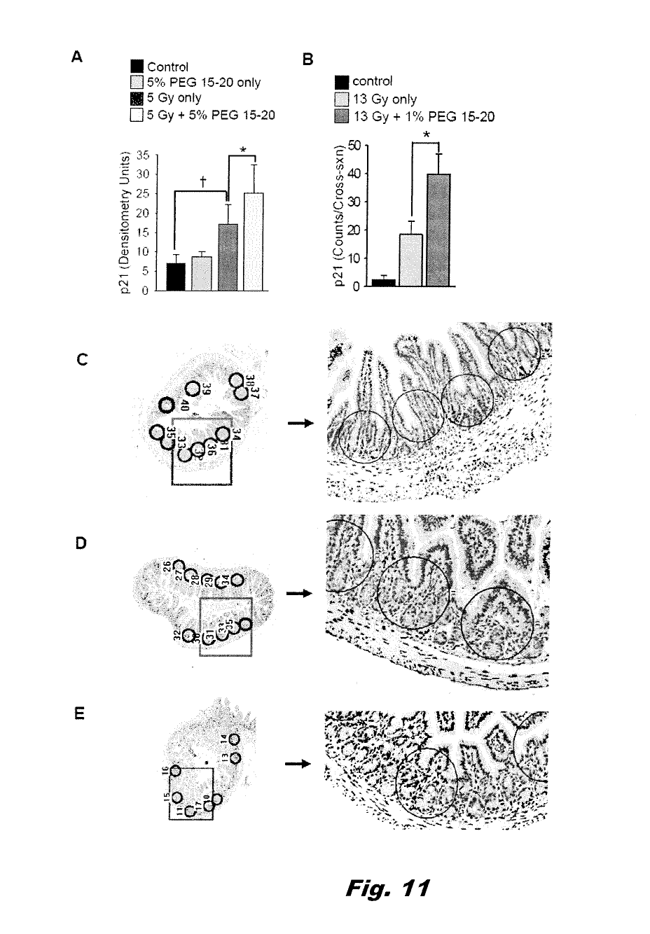

To determine if PEG 15-20 protection against radiation-induced apoptosis involved p53 and p21 pathways, reiterative in vitro and in vivo immunohistochemical analyses of both markers were performed on radiated IEC cells treated according to FIG. 9A and on ileal segments obtained 24 hours from non radiated control mice, mice subjected to abdominal radiation (13 Gy) drinking 5% dextrose alone, and in abdominally radiated mice drinking D5W+PEG 15-20. Radiated IEC cells demonstrated increased p53 levels, but surprisingly, PEG 15-20 resulted in a further and significant increase in p53 level both in cultured IEC cells and in mouse ileal segments (FIG. 10A-E). As increases in p53 can either protect against injury or trigger a proapoptotic pathway, reiterative studies were performed to assess the changes in p21 levels among the treatment groups. A p21 pattern similar to the p53 pattern was observed in response to PEG 15-20 both in vitro and in vivo, indicating that the protective effect of PEG 15-20 involved activation of p53 and p21 in a manner that leads to growth arrest and DNA repair (FIG. 11A-E).

Small Angle X-ray Scattering Analysis (SAXS) was used to determine how HMW PEG interacts with the phospholipid bilayer using modeled and fully ordered bilipid membranes. The SAXS pattern collected on samples prepared with 2 mol % PEG 15-20 at 40±2° C. shows two Bragg peaks positioned at q=0.114 and 0.226 Å⁻¹, indicative of 1-D lamellar (bilayer) stacks with a periodicity of 55.1 Å. This observation suggests that the HMW PEG is associated with the lipid bilayer in such a manner as to direct the two symmetric PEG blocks laterally across the membrane-water interface (not well projected from the surface), yielding a polymer coat across the lipid bilayer. Moreover, it was noted that the narrow diffraction peaks and the lack of any other features in the SAXS profile suggest the existence of a single phase, meaning that the polymer is, in fact, incorporated into the lipid mixture. The positioning of the HMW PEG laterally across the membrane-water interface is an ideal conformational state for barrier formation and protection of the extensive surface area of the intestinal mucosal.

Because HMW PEG incorporates into bilipid membranes, the effect of PEG on lipid raft coalescence, a morphological alteration know to occur following radiation exposure that play a critical role in cell apoptosis, was tested. Imaging of lipid raft distribution in control cells demonstrated a typical even distribution mostly concentrated in the membranes. Treatment of non-radiated control cells with 5% PEG altered lipid raft distribution to appear diffusely distributed and dispersed. When epithelial cells were radiated (5Gy) and exposed to P. aeruginosa, lipid raft coalescence was observed. Exposure of cells to P. aeruginosa or radiation alone also caused lipid raft coalescence but to a lesser degree. Pre-treatment with 5% PEG prior to radiation and exposure to P. aeruginosa prevented the observed coalescence and caused a dramatic dispersion pattern of fluorescence. Thus, the protective effect of PEG appeared to involve anti-apoptotic signaling via lipid rafts, p53 and p21. Specifically, lipid rafts are likely to be the major initiating target through which radiation injury and P. aeruginosa synergize to accelerate the process of cellular apoptosis and are also the likely site at which HMW PEGs exert their protective action.

Experimental results also established that PEG 15-20 maintains a protective shield on the apical surface of cultured intestinal epithelial cells and suppresses virulence expression in P. aeruginosa. PEG 15-20 can act as a surrogate mucin and prevent bacteria from invading the epithelial cell surface. Consistently, the protective effect of HMW-PEG against bacterial cell invasion was separately assayed using fluorescently labeled P. aeruginosa, with HMW-PEG reducing the level of P. aeruginosa invasion of epithelial cell barriers. In addition, HMW-PEG also appears to have a significant effect on the ability of P. aeruginosa to express virulence genes in response to quorum-sensing molecules when present in the lumen of the mouse intestine during surgical injury. To determine if these effects could be observed in the present model, the ability of PEG 15-20 to prevent bacterial invasion of IEC cells exposed to 5 Gy radiation was examined. Results demonstrated that the apical surfaces of radiated IEC cells became rapidly and densely colonized by P. aeruginosa compared to non-radiated (control) cells. When IEC cells were pre-treated with PEG 15-20 as outlined in FIG. 9A, P. aeruginosa maintained a visible distance from the apical surface of the cells, an effect that was observed for up to one hour (FIG. 12A). Finally, P. aeruginosa bacterial cells grown in the presence of PEG 15-20 were markedly attenuated in their ability to express PA-I lectin/adhesin, a potent epithelial barrier protein, as demonstrated in FIGS. 12B and 12C. Also, HMW PEG had no effect on bacterial growth rates.

In contrast to the teachings in the above-cited references that PEG of higher average molecular weight than PEG 600 had no effect in protecting cells against radiation, the studies disclosed herein establish that HMW PEG molecules have unique properties that facilitate their use in the mammalian gut as radioprotectants and as agents that preserve epithelial barrier function and cytoskeletal structure and protect against radiation-induced epithelial cell apoptosis.

### Example 9

### Methods of Monitoring Administration of HMW PEG

The invention also contemplates methods for monitoring administration of HMW PEG, e.g., in a method of treatment. In such monitoring methods, labeled HMW PEG is administered, alone or in combination with unlabeled HMW PEG, and the label is detected during treatment on a continuous or intermittent schedule, including simple endpoint determinations. The term “labeled” HMW PEG means that a label, or detectable compound, is directly or indirectly attached to HMW PEG, or the HMW PEG is attached to a reporter compound that is capable of associating a label with HMW PEG (of course, labels not attached to HMW PEG or designed to be associated therewith are also contemplated by the invention, as noted below). The HMW PEG is labeled using any detectable label known in the art, and the PEG is labeled to a level sufficient to detect it. Those of skill in the art will recognize that the level will vary depending on the label and the method of detection. One of skill in the art will be able to optimize the degree of labeling using routine optimization procedures. The label is chemically bound to the HMW PEG by a non-covalent or a covalent bond that is stable in use and, preferably, in storage. Label covalently bound to HMW PEG is preferred. The density of label attachment is adjusted to substantially preserve the biological activity of HMW PEG (preservation of sufficient biological activity to realize a beneficial prophylactic or therapeutic effect as disclosed herein). This is typically achieved by adjusting the HMW PEG:label ratio, as would be known in the art. Given the relative size of the average molecule of HMW PEG, it is expected that a wide variety of labels will be suitable for attachment to HMW PEG with substantial preservation of the biological activity thereof.

Labels contemplated by the invention are those labels known in the art, which include a radiolabel, a chromophore, a fluorophore, and a reporter (including an enzyme that catalyzes the production of a detectable compound and a binding partner such as an antibody that localizes a detectable compound in the vicinity of the reporter). Exemplary enzyme reporters include an enzymatic component of a luminescence system and a catalyst of a colorimetric reaction. More particularly, exemplary reporter molecules include biotin, avidin, streptavidin, and enzymes (e.g., horseradish peroxidase, luciferase, alkaline phosphatases, including secreted alkaline phosphatase (SEAP); β-galactosidase; β-glucuronidase; chloramphenicol acetyltransferase). The use of such reporters is well known to those of skill in the art and is described in, e.g., U.S. Pat. No. 3,817,837, U.S. Pat. No. 3,850,752, U.S. Pat. No. 3,996,345, and U.S. Pat. No. 4,277,437. Exemplary enzyme substrates, which may be converted to detectable compounds by reporter enzymes, include 5-bromo-4-chloro-3-indolyl β-D-galactopyranoside or Xgal, and Bluo-gal. Enzyme substrates, as compounds capable of conversion to detectable compounds, may also be labels in certain embodiments, as would be understood in the art. U.S. patents teaching labels, and their uses, include U.S. Pat. No. 3,817,837; U.S. Pat. No. 3,850,752; U.S. Pat. No. 3,939,350 and U.S. Pat. No. 3,996,345. Exemplary radiolabels are ³H, ¹⁴C, ³²P, ³³P, ³⁵S, and ¹²⁵I; exemplary fluorophores are fluorescein (FITC), rhodamine, Cy3, Cy5, aequorin, and green fluorescent protein. A preferred label is a fluorophore such as fluorescein.

The monitoring methods of the invention may also involve more than one label. In one embodiment, one label serves to identify the location of the HMW PEG following or during treatment, while a second label is specific for one or more microbes insofar as the label detectably associates with at least one microbe. For example, a monitoring method may include fluorescein attached to HMW PEG in a manner that substantially preserves the biological activity of the HMW PEG, and free (i.e., unattached) Xgal or bluo-gal for detection of prokaryote-specific β-galactosidase activity. The fluorescein localizes the HMW PEG, while a colored (blue) product indicates the presence of a lactose-metabolizing prokaryotic microbe, such as a Pseudomonad. The invention also includes monitoring methods wherein a single label provides this information (i.e., the location of HMW PEG and an indication of the presence of a microbe).

Any detection technique known in the art may be used in the monitoring methods of the invention. Several factors will influence the detection technique chosen, including the type of label, the biomaterial subjected to monitoring (e.g., epidermal cells of the skin, ear canal, or intestine; stool, mucus or tissue samples), the level of discrimination desired, whether quantitation is expected, and the like. Suitable detection techniques include simple visual inspection with the unaided eye, visual inspection with an instrument such as an endoscope, optionally equipped with a suitable light source and/or camera for recordation, the conventional use of Geiger counters, x-ray film, scintillation counters, and the like, and any other detection technique known in the art.

One of skill will recognize that the monitoring methods of the invention are useful in optimizing the treatment methods. For example, a monitoring method may be used to optimize the quantity and/or concentration of HMW PEG (e.g., to achieve a desired viscosity for a solution or mixture of HMW PEG), which is delivered to an epithelial cell, such as the epithelial cells of the ear canal to prevent or to treat swimmer's ear. By way of additional examples, optimization of bowel or intestinal treatments may be facilitated by endoscopic inspection of an intestinal tract exposed to labeled HMW PEG or by monitoring stool samples.

The monitoring methods of the invention include a stool assay for a microbe capable of adhering to an intestinal epithelial cell comprising contacting a microbe and an intestinal epithelial cell and detecting adherence of the microbe to the epithelial cell using any technique known in the art. In a preferred embodiment, the intestinal epithelial cell is immobilized on a suitable surface, such as the bottom and/or sides of a microtiter well. In another preferred embodiment, a direct label, or an indirect label such as a reporter capable of generating a detectable product, is added prior to, or during, the detecting step. The monitoring methods may further comprise addition of free label. For example, free Bluo-gal is added to a sample suspected of containing a lactose-metabolizing prokaryotic microbe; if present, the microbial enzyme β-galactosidase will cleave Bluo-gal to yield a detectable blue product.

In one embodiment, commercially available intestinal epithelial cells (e.g., Caco-2 cells, ATCC HTB 37, and/or IEC-6 cells, ATCC CRL 1952) are fixed to the wells of a microtiter dish using a conventional technique. A stool sample is collected and mixed with a fluid such as phosphate-buffered saline. The liquid phase of the mixture, containing suspended microbes, is obtained (e.g., by suitable filtration (i.e., separation of gross solids from bacteria in fluid suspension), decanting, or the like) and diluted 1:100 in PBS. Bluo-gal is added to the live microbial suspension. The microbial suspension is added to microtiter wells for 1 hour at 24° C., followed by washing of the wells with a suitable fluid (e.g., PBS) to remove unbound microbes. Microbes unbound and/or bound to the immobilized epithelial cells are detected, e.g., by counting using polarized light microscopy. In alternative embodiments, an immunoassay is used to detect adherence, with suitable immunological reagents being a microbe(s)-specific monoclonal or polyclonal antibody, optionally attached to a label such as a radiolabel, a fluorophore or a chromophore.

One of skill in the art will recognize that neither the intestinal epithelial cell nor the microbe is required to be immobilized, although such immobilization may facilitate accurate detection of microbes adhering to epithelial cells. For example, in one embodiment, an immobilized stool microbe is brought into contact with an intestinal epithelial cell that is not immobilized. Further, one of skill would recognize that any suitable fluid known in the art may be used to obtain the microbial suspension, with preferred fluids being any of the known isotonic buffers. Also, as noted above, any known label may be used to detect cell adherence.

In a related aspect, the invention provides a kit for assaying for microbial cell adherence comprising an epithelial cell and a protocol for assaying microbial cell adherence to the epithelial cell. The protocol describes a known method for detecting a microbe. A preferred kit includes an intestinal epithelial cell. Other kits of the invention further comprise a label, such as a fluorophore or a reporter.

Another monitoring method contemplated by the invention is an assay for microbial hydrophobicity. In this method, the relative or absolute hydrophobicity of a microbial cell is determined using any conventional technique. An exemplary technique involves exposure of any microbe to hydrophobic interaction chromatography, as would be known in the art. Ukuku et al., J. Food Prot. 65:1093-1099 (2002), incorporated herein by reference in its entirety. Another exemplary technique is non-polar:polar fluid partition (e.g., 1-octanol:water or xylene:water) of any microbe. See Majtan et al., Folia Microbiol (Praha) 47:445-449 (2002), incorporated herein by reference in its entirety.

In one embodiment of a hydrophobicity assay for monitoring PEG administration, a stool sample is suspended in 50 mM sodium phosphate buffer (pH 7.4) containing 0.15 M NaCl. Microbes in the suspension are collected by centrifugation and resuspended in the same buffer, and the centrifugation-resuspension cycle is repeated. If feasible, the microbes are resuspended in the same buffer to an absorbancy of 0.4 at 660 nm, which will permit monitoring spectrophotometrically, without using labeled PEG. The microbial suspension is treated with xylene (2.5:1, v/v, Merck), the suspension is vigorously mixed for two minutes, and the suspension is allowed to settle for 20 minutes at room temperature. The presence of microbes in the aqueous phase is then determined, for example by spectrophotometric determination of absorbancy at 660 nm. A blank containing the sodium phosphate buffer is used to eliminate background.

In obtaining microbial cells from stool samples for use in these methods, it is preferred that the HMW PEG be relatively insoluble in the fluid used to obtain the microbial suspension and any fluid used to dilute the microbial suspension.

The invention further provides a kit for performing the monitoring method comprising an assay for microbial hydrophobicity, which comprises an intestinal epithelial cell and a protocol describing the determination of microbial hydrophobicity. A preferred kit includes an intestinal epithelial cell. Related kits further comprise a label, such as a fluorophore or a reporter.

Still further, the invention provides a monitoring method comprising obtaining a sample of intestinal flora and detecting PA-I lectin/adhesin activity. Any technique for detecting PA-I lectin/adhesin activity known in the art may be used. For example, PA-I lectin/adhesin may be detected using an antibody (polyclonal, monoclonal, antibody fragment such as a Fab fragment, single chain, chimera, humanized or any other form of antibody known in the art) that specifically recognizes PA-I lectin/adhesin. The immunoassay takes the form of any immunoassay format known in the art, e.g., ELISA, Western, immunoprecipitation, and the like. Alternatively, one may detect a carbohydrate-binding capacity of PA-I lectin/adhesin or the intestinal epithelial barrier breaching activity of PA-I lectin/adhesin may be measured, e.g., by monitoring the trans-epithelial electrical resistance or TEER of an epithelial layer prior to, and/or during, exposure to a sample. In related kits, the invention provides a PA-I lectin/adhesin binding partner and a protocol for detecting PA-I lectin/adhesin activity (e.g., binding activity). Other kits according to the invention include any carbohydrate known to bind PA-I lectin/adhesin and a protocol for detecting PA-I lectin/adhesin activity (e.g., binding activity).

### Example 10

### HMW PEG Induces Epithelial Monolayer Injury Repair

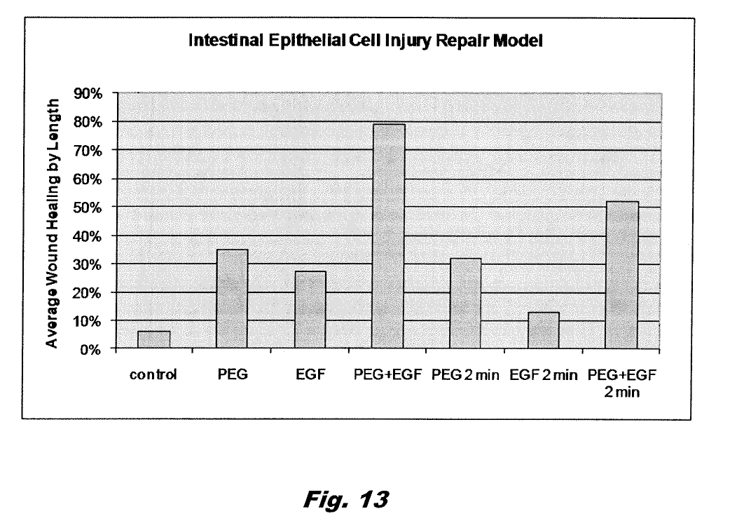

In order to assess the ability of HMW PEG to induce the repair of epithelial monolayers following injury, IEC-18 cells were grown to confluency in 60 mm dishes, rinsed then the monolayer was mechanically wounded using a razor blade. Detached cells were removed and fresh medium containing 5% PEG 15-20, 50 ng/ml Epidermal Growth Factor (EGF) or a combination of both was added. The cells were maintained in the presence of treatment for 24 hours or were treated for 2 minutes, washed then incubated without treatment for 24 hours. Wound healing was assessed by the decrease in the length of the wound after 24 hours. As shown in FIG. 13, both HMW PEG and EGF induced wound healing individually and the combination of HMW PEG and EGF resulted in a synergistic induction of this effect. Although this assay measures the healing of mechanically induced intestinal epithelial wounds, it is expected that a similar effect would be observed for epithelial wounds caused by other insults, such as radiation-induced cell death.

Numerous modifications and variations of the present invention are possible in view of the above teachings and are within the scope of the invention. The entire disclosures of all publications cited herein are hereby incorporated by reference.

What is claimed is:

1. A use of a high molecular weight polyethylene glycol (HMW PEG) compound in the preparation of a medicament for treating a mammalian epithelial cell exposed to radiation.

2. The use according to claim 1 wherein the mammalian epithelial cell is further exposed to an intestinal pathogen.

3. The use according to claim 2 wherein the intestinal pathogen is a Pseudomonad.

4. The use according to claim 3 wherein the intestinal pathogen is Pseudomonas aeruginosa.

5. The use according to claim 1 wherein the HMW PEG compound has an average molecular weight selected from the group consisting of at least 1,000 daltons, at least 5,000 daltons, at least 8,000 daltons, at least 12,000 daltons and at least 15,000 daltons.

6. The use according to claim 5 wherein the HMW PEG comprises at least two hydrocarbon chains attached to a hydrophobic core, wherein each hydrocarbon chain has an average molecular weight of at least 40 percent of the HMW PEG compound, and wherein the hydrophobic core comprises a ring structure.

7. A use of a high molecular weight polyethylene glycol (HMW PEG) compound in the preparation of a medicament for treating a microbial pathogen-induced disorder of an irradiated mammalian epithelial cell.

8. The use according to claim 7 wherein the microbial pathogen is Pseudomonas aeruginosa.

9. The use according to claim 8 wherein the HMW PEG has an average molecular weight selected from the group consisting of at least 1,000 daltons, at least 5,000 daltons, at least 8,000 daltons, at least 12,000 daltons and at least 15,000 daltons

10. A use of a high molecular weight polyethylene glycol (HMW PEG) compound in the preparation of a medicament for treating a mammalian epithelial cell in combination with radiotherapy.

11. The use according to claim 10 wherein the HMW PEG has an average molecular weight selected from the group consisting of at least 1,000 daltons, at least 5,000 daltons, at least 8,000 daltons, at least 12,000 daltons and at least 15,000 daltons.

12. A use of a high molecular weight polyethylene glycol (HMW PEG) compound in the preparation of a medicament for protecting a mammalian epithelial cell from radiation-induced damage.

13. The use according to claim 12 wherein the HMW PEG compound has an average molecular weight selected from the group consisting of at least 1,000 daltons, at least 5,000 daltons, at least 8,000 daltons, at least 12,000 daltons and at least 15,000 daltons.

14. A use of a high molecular weight polyethylene glycol (HMW PEG) compound in the preparation of a medicament for protecting an irradiated mammalian epithelial cell from microbial pathogen-induced damage.

15. The use according to claim 14 wherein the HMW PEG compound has an average molecular weight selected from the group consisting of at least 1,000 daltons, at least 5,000 daltons, at least 8,000 daltons, at least 12,000 daltons and at least 15,000 daltons.

16. The use according to claim 14 wherein the microbial pathogen is Pseudomonas aeruginosa.

17. A use of a high molecular weight polyethylene glycol compound in the preparation of a medicament for preventing sepsis in a mammal having abdominal radiation.
